(19) United States
(12) Patent Application Publication
(10) Pub. No.: US 2011/0212153 A1
(43) Pub. Date: Sep. 1, 2011

(54) COMPOSITE COATINGS AND DEPOSITION METHODS

(76) Inventors: Jayesh R. Bellare, Mumbai (IN); Smriti Sharma, Sunnyvale, CA (US); Vivek P. Soni, Maharashtra (IN)

(21) Appl. No.: 12/715,267

(22) Filed: Mar. 1, 2010

(51) Int. Cl. A61K 9/00; C25D 13/06; B32B 17/06; A61P 19/00

(52) U.S. Cl. 424/423; 204/484; 428/426

(57) ABSTRACT

Provided in part herein are methods for preparing coated support materials and medical implants. Also provided in part herein are coated support materials and medical implants. Provided also in part herein are methods for treating injuries using coated support materials and medical implants.

### FIELD

The technology relates in part to biocompatible composite coatings suitable for use with medical implants.

### BACKGROUND

Medical implants, and in particular implants used in bone repair and/or joint replacement, often are made of a rigid material, such as a metal, for example. Metals often are chosen for their strength, durability and load bearing capability. Biocompatible materials can be used to coat medical implants to enhance implant/host cell interactions. Biocompatible materials sometimes are derived from organic materials and sometimes are derived from inorganic materials.

### SUMMARY

Provided in part herein is a method, including: contacting a support material with a bioactive polymer under a first set of electrophoresis conditions that include applying an electric current of less than 5 mA/cm², where the bioactive polymer is deposited onto the support material; contacting the support material with bioceramic particles under a second set of electrophoresis conditions, where the bioceramic particles are deposited onto the support material; and alternating the depositing of the bioactive polymer and the bioceramic particles onto the support material in a predetermined number of cycles, where a coated support material is prepared.

Also provided in part herein is a coated solid material, including a support material that incorporates an exterior surface and a coating adhered to the exterior surface that includes a bioactive polymer and bioceramic particles, where the coated solid material is prepared by a method including: contacting a support material with a bioactive polymer under a first set of electrophoresis conditions that include applying an electric current of less than 5 mA/cm², where the bioactive polymer is deposited onto the support material; contacting the support material with bioceramic particles under a second set of electrophoresis conditions, where the bioceramic particles are deposited onto the support material; and alternating the depositing of the bioactive polymer and the bioceramic particles onto the support material in a predetermined number of cycles, where a coated support material is prepared. The exterior surface coating generally includes the bioactive material and the bioceramic material.

Provided also in part herein is a coated material (e.g., solid material), including: a support material having an exterior surface, a coating adhered to the exterior surface, and cells in association with the coated material, where: the coating includes a polysaccharide, bioceramic particles or a polysaccharide and bioceramic particles. The polysaccharide sometimes contains glucose, and the bioceramic particles sometimes include apatite and wollastonite.

Also provided in part herein is a coated medical implant, including: a support material having an exterior surface, a coating adhered to the exterior surface, and cells in association with the coated medical implant, where the coating includes a polysaccharide, bioceramic particles or a polysaccharide and bioceramic particles. The polysaccharide sometimes contains glucose, and the bioceramic particles sometimes include apatite and wollastonite.

Provided also in part herein is a method, including: contacting a coated material (e.g., solid material) with cells under cell association conditions, where the cells adhere to the coated solid material and where: the coated solid material includes a support material having an exterior surface and a coating adhered to the exterior surface, the coating includes a polysaccharide, and the coating includes bioceramic particles.

Also provided in part herein is a method, including: inserting a coated implant into a subject, whereby the coated implant is fused with vasculature in the subject after a period of time, where: the coated implant includes a support material having an exterior surface and a coating adhered to the exterior surface, the coating includes a polysaccharide, and the coating includes bioceramic particles.

In certain embodiments, the bioactive polymer and bioceramic particles are deposited in layers. In some embodiments, the combination of bioactive polymer and bioceramic particles include a bioactive material. In certain embodiments, the coating includes layers. In some embodiments each cycle is repeated between 2 and about 1000 times. In certain embodiments, the layers independently include a polysaccharide or bioceramic particles. In some embodiments, all or some of the bioactive polymer includes a positive charge at physiological pH, and sometimes the bioactive polymer has a pK property of about 4 to about 7. In some embodiments, the polysaccharide includes glucose. In certain embodiments, the polysaccharide includes a 2-amino-2-D-glucose polymer. In certain embodiments, the polymer includes chitosan. In some embodiments, the bioceramic particles include apatite. In certain embodiments, the bioceramic particles include wollastonite. In some embodiments, the bioceramic particles contain apatite and wollastonite. In certain embodiments, apatite and wollastonite particles are in the form of a powder prior to deposition. In some embodiments, the powder is manufactured from a sol-gel precursor. In certain embodiments, the powder is manufactured by sintering a precursor (e.g., a sol-gel precursor).

In some embodiments, the bioceramic particles are about 50 to about 500 nanometers in diameter. In certain embodiments, bioceramic particles are about 200 nanometers in diameter. In some embodiments, the layers have a thickness of about 0.1 micrometers to about 100 micrometers. In some embodiments, the coating is about 5 micrometers to about 500 micrometers thick. In certain embodiments, the outermost layer includes the bioactive polymer.

In some embodiments, the support material includes a metal. In certain embodiments, the metal includes titanium. In some embodiments, the metal includes a titanium alloy. In certain embodiments, the metal includes steel. In some embodiments, the metal includes a steel alloy. In certain embodiments, the support material is etched, and in some embodiments, the support is etched with an acid.

In certain embodiments, the support material is a medical implant. In some embodiments, the implant is a hip joint implant. In certain embodiments, the implant is a knee joint implant. In some embodiments, the implant is an orthopedic medical implant. In certain embodiments, the medical implant is a dental implant. In some embodiments, the bioactive material adheres to cells. In certain embodiments, the coated solid material further includes cells adhered to the coated support material.

In certain embodiments, the associated cells are mammalian cells. In some embodiments, the cells are derived from bone, form bone, or are derived from bone and form bone. In certain embodiments, the cells are derived from cartilage, form cartilage, or are derived from cartilage and form cartilage. In some embodiments, the cells are derived from muscle, form muscle, or are derived from muscle and form muscle. In certain embodiments, the cells are derived from connective tissue, form connective tissue, or are derived from connective tissue and form connective tissue. In some embodiments, the cells are derived from vasculature, form vasculature, or are derived from vasculature and form vasculature. In some embodiments, the coated support material is contacted with cells selected from one or more of stem cells, embryonic cells, primordial cells, partially differentiated cells or differentiated cells. In certain embodiments, the cells are in a vasculature structure. In some embodiments, methods described herein include contacting the coated support material with cells under cell association conditions, and in some embodiments, the methods include implanting the coated support material having adhered cells into a subject. In certain embodiments, cells are not in association with the coated medical implant.

In some embodiments, electrophoresis under the first electrophoresis conditions, is carried out for less than about 5 minutes (e.g., about 10 seconds, about 30 seconds, about 1 minute, about 90 seconds, about 2 minutes, about 3 minutes, about 4 minutes, about 270 seconds). In certain embodiments, electrophoresis is carried out for less then about 3 minutes (e.g., about 10 seconds, about 30 seconds, about 1 minute, about 90 seconds, about 2 minutes, about 150 seconds). In some embodiments, electrophoresis is carried out for between about 30 seconds and about 90 seconds. In certain embodiments, electrophoresis under the first set of electrophoresis conditions, is carried out at a constant current. In some embodiments, the current is less than about 4 mA/cm². In certain embodiments, the current is less than about 3 mA/cm². In some embodiments, the current is less than about 2 mA/cm². In some embodiments, the current is about 1 mA/cm².

In certain embodiments, electrophoresis under the second electrophoresis conditions is carried out for over 5 minutes (e.g., about 330 seconds, 6 minutes, 390 seconds, 7 minutes, 450 seconds, 8 minutes, 510 seconds 9 minutes, 570 seconds, 10 minutes). In some embodiments, electrophoresis is carried out for between 6 and 8 minutes (e.g., about 6 minutes, 390 seconds, 7 minutes, 450 seconds, 8 minutes). In certain embodiments, the electrophoresis is carried out for more than 10 minutes. In certain embodiments, electrophoresis under the second set of electrophoresis conditions is carried out at a constant current. In some embodiments, the current is less than about 4 mA/cm². In certain embodiments, the current is between about 2 mA/cm² and about 4 mA/cm². In some embodiments, the current is about 3 mA/cm².

In certain embodiments, methods described herein include contacting the coated support material with a biologically active additive under deposition conditions and depositing the biologically active additive onto the coated support material. In some embodiments, the biologically active additive is selected from bio-nutrients, antibiotics, growth factors, hormones, drugs, and the like, or combinations thereof. In certain embodiments, the biologically active additive is deposited as a last step in preparing a medical implant device.

The foregoing summary illustrates certain embodiments and does not limit the disclosed technology. In addition to illustrative aspects, embodiments and features described above, further aspects, embodiments, and features will become apparent by reference to the drawings and the following detailed description and examples.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 show representative images of coating depositions performed at different pHs. FIG. 2 show representative images of coating depositions performed at different current densities. FIG. 3 illustrates the X-ray diffraction patterns of apatite-wollastonite powder and composite coating (titanium peaks removed from composite coating pattern).

FIGS. 4A-4C show representative images of a composite coating embodiment, as described herein, using different magnifications and viewing methods. FIG. 4A is a representative image of a scanning electron micrograph (SEM) top view image of a composite coating as described herein, where the scale bar is roughly equivalent to about 10 micrometers. FIG. 4B is a representative image of a SEM image of a cross sectional view of a composite coating as described herein, where the scale bar is roughly equivalent to about 5 micrometers. FIG. 4C is a back scatter electron micrograph image with compositional contrast (BSE COMPO), where the scale bar is roughly equivalent to about 5 microns. FIG. 5 shows a representative load-displacement curve of a composite coating prepared as described herein.

FIGS. 6A-6F show scanning electron micrograph (SEM) images and energy dispersive X-ray spectroscopy (EDAX) of composite coatings embodiments, as described herein, immersed in synthetic body fluid (SBF). Scale bars in FIGS. 6A-6C are roughly equivalent to about 10 micrometers. The white rectangular outline in FIGS. 6A-6C indicate the localized area in which spectroscopy was performed. FIG. 6A is an SEM image of a coating immersed in SBF for 7 days. FIG. 6B is an SEM image of a coating immersed in SBF for 14 days. FIG. 6C is an SEM image of a coating immersed in SBF for 21 days. FIG. 6D shows the spectroscopic peaks of various elements in the localized region of the coating showing in FIG. 6A, after immersion in SBF for 7 days. FIG. 6E shows the spectroscopic peaks of various elements in the localized region of the coating showing in FIG. 6B, after immersion in SBF for 14 days. FIG. 6F shows the spectroscopic peaks of various elements in the localized region of the coating showing in FIG. 6C, after immersion in SBF for 21 days.

FIG. 7 shows an SEM image of apatite-wollastonite coating (e.g., ceramic only, not a composite coating) immersed in SBF for 14 days showing ball-like apatite grown, where the scale bar is roughly equivalent to 10 micrometers. FIGS. 8A-8D illustrate a proposed mechanism for apatite growth on the composite coating embodiments described herein. Steps 1-4 of the proposed mechanism (e.g., FIGS. 8A-8D, respectively) are described further in the Examples. FIG. 9 shows the Raman spectra of control and coated samples immersed in SBF for 7 days, 14 days, and 21 days.

FIGS. 10A-10D show representative images of bones treated with implants 14 days and 21 days post insertion in a subject. FIGS. 10A and 10C illustrate gross observations on implants coated with a composite coating embodiment, as described herein, inserted in a subject for 14 days and 21 days respectively. FIGS. 10B and 10D illustrate gross observations on uncoated implants inserted in a subject for 14 days and 21 days respectively.

FIGS. 11A-11D show representative images of bones treated with implants 35 days and 42 days post insertion in a subject. FIGS. 11A and 11C illustrate gross observations on implants coated with a composite coating embodiment, as described herein, inserted in a subject for 35 days and 42 days, respectively. FIGS. 11B and 11D illustrate gross observations on uncoated implants, inserted in a subject for 35 days and 42 days, respectively.

FIG. 12A-12D show representative images of radiographs of bones treated with implants 14 days and 21 days post insertion in a subject. FIGS. 12A and 12C show radiographs of bones treated with an implant coated with a composite coating embodiment, as described herein, inserted into a subject at 14 days and 21 days, respectively. FIGS. 12B and 12D show radiographs of bones treated with uncoated implants inserted into a subject at 14 days and 21 days, respectively.

FIG. 13A-13D show representative images of radiographs of bones treated with implants 35 days and 42 days post insertion in a subject. FIGS. 13A and 13C show radiographs of bones treated with an implant coated with a composite coating embodiment, as described herein, inserted into a subject at 35 days and 42 days, respectively. FIGS. 13B and 13D show radiographs of bones treated with uncoated implants inserted into a subject at 35 days and 42 days, respectively.

FIGS. 14A-14D, 15A and 15B show representative images of histopathological slides prepared from host bone at or near the site of implant insertion 14 days, 21 days, 35 days and 42 days after insertion of coated or uncoated implants. The scale bars in FIGS. 14A, 14B, and 14D are roughly equivalent to 100 micrometers. The scale bars for FIGS. 14C, 15A and 15B are roughly equivalent to 50 micrometers. FIGS. 14A and 14C show bone organization at or near the insertion site of an implant treated with a composite coating embodiment, as described herein, after 14 days and 21 days, respectively. FIGS. 14B and 14D show bone organization at or near the insertion site of an uncoated implant after 14 days and 21 days, respectively. FIGS. 15A and 15B show bone organization at or near the insertion site of an implanted treated with a composite coating embodiment, as described herein, after 35 days and 42 days, respectively. Diffuse infiltration of resting cartilage (RC) can be seen around day 14 at sites treated with coated implants, indicating the onset of osteogenesis (see FIG. 14A), while sites treated with uncoated implants show a moderate infiltration of fibrous connective tissue (FCT), around day 14 (see FIG. 14B). Around day 21, multifocal foci of hypertrophy of chondrocytes (HC) and bone trabeculae (BT) can be seen in sites treated with coated implants. Multifocal HC generally are indicative of stacking of chondrocytes, which in turn can lead to calcification of chondrocytes (CC) as seen in FIGS. 14A and 14C. In FIG. 14C (e.g., day 21), extensive calcification and formation of BT indicated bone ossification. Sites treated with uncoated implants continue to show HC and FCT, with only non-multifocal foci visible around day 21. No BT and little or no CC is observed around day 21 in sites treated with uncoated implants. Around day 35, lamellar bone (LB), bone marrow (BM) and the proliferation of blood vessels is seen in sites treated with coated implants (see FIGS. 15A and 15B). In FIG. 15B (e.g., day 42), extensive LB and bone remodeling can be seen. Additional discussion of FIGS. 14A-15B is provided in Example 2. FIGS. 16A-16D show representative images of fluorescence imaging new bone growth at implant insertion sites in subjects treated with coated or uncoated implants. The scale bars in FIGS. 16A-16D is roughly equivalent to about 100 micrometers. FIGS. 16A and 16C show new bone growth near the site of insertion in subjects treated with an implant having a composite coating embodiment described herein after 21 days and 42 days, respectively. FIGS. 16B and 16D show new bone growth near the site of insertion in subjects treated with an uncoated implant.

### DETAILED DESCRIPTION

In the following detailed description, reference is made to the accompanying drawings, which form a part hereof. In the drawings, similar symbols typically identify similar components, unless context dictates otherwise. Illustrative embodiments described in the detailed description, drawings, and claims do not limit the technology. Other embodiments may be utilized, and other changes may be made, without departing from the spirit or scope of the subject matter presented herein. It will be readily understood that aspects of the present disclosure, as generally described herein, and illustrated in the drawings, can be arranged, substituted, combined, separated, and designed in a wide variety of different configurations, all of which are explicitly contemplated herein.

Medical and dental procedures sometimes include implantation of a support material into a subject to aid in treatment of damaged or non-functional bone, cartilage or tissue. Support materials used for implants in medical or dental procedures frequently are manufactured from materials chosen for their durability, strength, and/or load bearing capacity. Metals sometimes are chosen for support materials used as implants due to their favorable characteristics with regard to durability, strength and load-bearing capacity.

Some materials chosen for support materials, including many metals, have reduced interaction with the host biological environment, or are biologically inert, and therefore do not readily interact with the tissue of the subject receiving the implant. Biologically inert materials sometimes cause zones of tissue avoidance or tissue rejection in the vicinity of the inserted support material. Non-limiting example of materials having reduced interaction with the host biological environment and/or that are biologically inert include certain metals, certain glasses or ceramics and certain plastics. Metals and other materials that are biologically inert or have reduced interaction with the biological environment can be coated with biocompatible or bioactive materials (e.g., composite coatings as described herein) to enhance subject tissue/implant interaction, thus enhancing the healing process by contributing to a reduction in recovery time.

Support Materials

Support materials (e.g., medical implants) sometimes are used to support, repair, enhance or replace damaged tissue in a subject. Support materials sometimes provide a protective housing or environment for implants that include electronic medical devices inserted into a subject. Non-limiting examples of implants include bone plates, screws, pins, joints, pacemakers, stents, cochlear implants, corneas, artificial vasculature, heart valves, dentures, dental bridges, prosthetics (e.g., maxillofacial prosthetics), artificial connective tissue, skin repair devices, pumps, catheters, bone cement and the like. In some embodiments, an implant is a hip joint implant. In certain, embodiments, an implant is a knee joint implant. Implants sometimes also provide additional functions. Non-limiting examples of additional functions sometimes provided by support materials include drug (e.g., drug delivery), bioactive material or bionutrient delivery; electrical stimulation; focusing (e.g., cornea), and the like, and combinations thereof.

Support materials often are made of a solid material (e.g., completely solid or partially solid (e.g., porous)), that includes an exterior surface. Support materials can be manufactured from any suitable material. Non-limiting examples of materials from which medical implants can be manufactured include plastics, polymers, metals, ceramics, carbon fiber, and the like, and combinations thereof. In some embodiments, a support material includes a metal. Non-limiting examples of metals suitable for manufacture of medical implants include titanium, titanium alloys, cobalt, cobalt alloys, steel, steel alloys, and the like, and combinations thereof. In certain embodiments, a support material includes titanium, and in some embodiments, a support material includes a titanium alloy. In certain embodiments, a support material includes steel, and in some embodiments, a support material includes a steel alloy. In certain embodiments, the support material is a medical implant.

The exterior surface of support materials can form an association with the tissue or cells of a subject, in some embodiments. Medical implants made of materials that are biologically inert or that have reduced biological interaction (titanium and titanium alloys, for example) can be surface treated, in certain embodiments. Non-limiting examples of surface treatments include etching (e.g. mechanical or chemical), coating (e.g., plasma coating, electrophorectic deposition, dipping), heating (e.g., sintering), and the like, and combinations thereof. In some embodiments, the surface of a support material is etched (e.g., etched with a chemical (e.g., acid), etched mechanically). In certain embodiments, the surface of a support material is coated. In some embodiments, the surface of a support material is etched and coated. In certain embodiments, the surface of a support material or medical implant is prepared by etching prior to coating.

Etching sometimes is used to prepare a surface for further treatment. Etching is a process where the surface of an object or material is scored, cut or abraded using mechanical force, electricity, chemicals, light, and the like, and combinations thereof. In some embodiments, the support material is etched with an acid. Any suitable acid can be used to surface treat or etch a support material. Non-limiting examples of acids suitable for etching a metallic support material are known and include hydrochloric acid, hydrofluoric acid, sulfuric acid, and the like. Non-limiting examples of etching conditions for titanium are described in the Examples.

In certain embodiments, treatment of the surface of a solid support material enhances biocompatibility (e.g., facilitates, enhances or improves implant/host cell interaction as compared to an untreated solid material). In certain embodiments, treatment (or further treatment) of the surface of a solid support material includes coating the surface of the solid support material with a biocompatible material.

Coatings

Biocompatible and/or bioactive materials can be coated on medical implants or solid support materials to enhance implant/host tissue or host cell interaction. Coated medical implants or solid support materials can decrease the recovery time associated with certain medical or dental procedures when compared to non-coated implants or support materials, in some embodiments. Biocompatible and/or bioactive materials can be obtained and/or derived from a variety of organic and inorganic sources.

The terms “biocompatible material”, “biocompatible materials”, “bioactive material”, “bioactive materials”, “biologically active compounds” and “biologically active molecules,” and grammatical variants (collectively “bioactive material”), as used herein, refer to materials, molecules or compounds, synthetic or naturally occurring, that can; (i) associate with living cells or tissue; (ii) have an effect on, or cause a reaction in living cells or tissue; (iii) function in contact with living tissue; and/or (iv) replace or repair part or all of a living system, for example. A bioactive material sometimes exhibits one or more of the following properties: charged or partially charged at physiological pH (e.g., positive charge, negative charge, pKa of about 3 to about 7, pKa of about 8 to about 11), biocompatible, biodegradable, haemostatic and anti-bacterial. Bioactive materials can be used to interface with biological systems to evaluate, treat, augment or replace any tissue, organ or function of the body. Bioactive materials suitable for one type of biological application sometimes are not suitable for other types of biological applications, and suitable applications for each type of biocompatible material are known. In some embodiments, a bioactive material forms a contact or association with the surrounding tissue in a suitable time and does not lead to rapid resorption (e.g., is not resorbed before tissue association and/or healing are substantially complete). In certain embodiments, the bioactive material adheres to cells. In some embodiments, a bioactive material associates with an extracellular matrix.

Any suitable bioactive material can be used to coat the surface of a partially or completely solid support material or medical implant. Non-limiting examples of bioactive and/or biocompatible materials suitable for use as a coating on medical implants include bioactive polymers, bioactive ceramics, biomimetic materials (e.g., polyvinyl alcohols, laminin, fibronectic, extra-cellular matrix proteins, and the like, and combinations thereof), biologically active molecules and compounds (e.g., biologically active additives), and the like, and combinations thereof. The term “biomimetic materials”, as used herein, refers to materials, naturally occurring or synthetic, adapted or developed using inspiration from nature and/or adapted or developed to mimic a naturally occurring biological structure. Some biomimetic materials can be configured to elicit specific cellular responses.

Biologically Active Polymers

Biologically active polymers (also referred to as bioactive polymers or biopolymers, for example, and used interchangeably throughout the document), can be naturally occurring, prepared from naturally occurring sources, or manufactured synthetically. Non-limiting examples of bioactive polymers include polysaccharides (e.g., glucose, chitin), polyanhydrides, polyamines, peptides, proteins, nucleic acids, cellulose, starch, dendrimers, lignin-based materials, synthetic bioactive polymers, and the like, and combinations thereof. A non-limiting example of a bioactive polymer that is prepared from a naturally occurring source is the preparation of chitosan from chitin.

Chitin is an abundant, naturally occurring long-chain polymer of N-acetylglucosamine (N-acetyl-D-glucos-2-amine), a derivative of glucose. Chitin is the main component of the cell walls of fungi, the exoskeletons of arthropods such as crustaceans (e.g. crabs, lobsters and shrimps) and insects and is found in a number of other organisms. In nature, chitin performs similar functions as cellulose (e.g., a polysaccharide) and keratin (e.g., a protein). Chitosan can be prepared from chitin by deacetylation.

Chitosan is a linear polysaccharide composed of randomly distributed β-(1-4)-linked D-glucosamine (deacetylated unit) and N-acetyl-D-glucosamine (acetylated unit). The degree of deacetylation (% DD) can be determined by Nuclear Magnetic Resonance (NMR) spectroscopy, and the % DD in commercial chitosan preparations often is in the range of 60% to 100%. The amino group in chitosan has a pKa value of approximately 6.5, making chitosan positively charged and soluble in acidic to neutral solution with a pH dependent charge density.

Chitosan sometimes functions as a bioadhesive which readily binds to negatively charged surfaces or molecules. In some embodiments, a bioactive polymer material includes a polysaccharide. In certain embodiments, the polysaccharide includes glucose. In some embodiments, the polysaccharide includes a 2-amino-2-D-glucose polymer. In certain embodiments, the polysaccharide includes chitosan.

Bioceramics

Biologically active ceramics (also referred to as “bioactive ceramics” or “bioceramics,” for example, and used interchangeably through the document), generally are ceramic materials that are biocompatible and include bioglasses and glass-ceramics. Ceramic materials often are inorganic, non-metallic materials formed from the action of heat and subsequent cooling. In some embodiments, ceramics can include metal oxides. Ceramics can be crystalline or non-crystalline in nature. Bioceramics sometimes are available as sol-gel and sometimes are available as a powder or particles.

The term “sol-gel” as used herein, refers to a chemical solution deposition technique often used in ceramics engineering. A “sol-gel process” often is used for the fabrication of materials (typically a metal oxide) starting from a chemical solution that acts as the precursor for an integrated network (or gel) of discrete particles or network polymers. The precursor sol can be used to synthesize powders (e.g., microspheres, nanospheres, microparticles, nanoparticles), deposited on a substrate to form a film (e.g., by dip coating or spin coating), or cast into shapes using a suitable container with the desired shape (e.g., thereby generating monolithic ceramics, glasses, fibers, membranes, aerogels and the like, in a desired shape). In some embodiments, bioceramic particles are manufactured from a sol-gel precursor. In certain embodiments, bioceramic particles are manufactured by a process including sintering. In some embodiments, bioceramic particles are manufactured by a process including ball milling. In certain embodiments, bioceramic particles are manufactured by a process including ball milling and sintering.

Bioceramics typically are categorized by their bioactivity which ranges from bioinert (e.g., non-toxic, non-inflammatory) to bioactive (e.g., associates with cells, elicits a favorable cellular response, or is resorbed by the host). Bioceramics frequently are coated on joint replacement medical implants to reduce wear and inflammatory responses. Non-limiting examples of bioinert bioceramics include oxide ceramics, silica ceramics, carbon fiber, synthetic diamond and the like. Non-limiting examples of bioactive bioceramics include bioactive glass (e.g., 45S5 Bioglass™), hydroxyapatite (hydroxylapatite), wollastonite, porous ceramics, porcelin, resorbable calcium phosphates, glass-ceramics, and the like, and combinations thereof. In some embodiments, bioceramic particles include apatite. In certain embodiments, bioceramic particles include wollastonite. In some embodiments, bioceramic particles include apatite and wollastonite. In certain embodiments, the bioceramic particles are about 50 nanometers to about 500 nanometers in diameter (e.g., about 50 nanometers, about 55 nanometers, about 60 nanometers, about 65 nanometers, about 70 nanometers, about 75 nanometers, about 80 nanometers, about 85 nanometers, about 90 nanometers, about 95 nanometers, about 100 nanometers, about 125 nanometers, about 150 nanometers, about 175 nanometers, about 200 nanometers, about 225 nanometers, about 250 nanometers, about 275 nanometers, about 300 nanometers, about 325 nanometers, about 350 nanometers, about 375 nanometers, about 400 nanometers, about 425 nanometers, about 450 nanometers, about 475 nanometers, or about 500 nanometers). In some embodiments, the bioceramic particles are about 200 nanometers in diameter.

Deposition Methods and Conditions

Biocompatible or bioactive composite coatings often are formed by coating objects (e.g., medical implants, support materials) with a combination of biocompatible materials. Any suitable biocompatible material can be used to generate a biocompatible composite coating. Biocompatible materials such as chitosan and apatite-wollastonite can be coated onto a medical implant, thereby generating a biocompatible composite coating, in some embodiments.

Composite coatings can be deposited on medical implants using a variety of deposition methods. Non-limiting examples of deposition methods include electrophoretic deposition and plasma spraying. Electrophoretic deposition offers advantages in cost of equipment and materials, and can be done at room temperature and atmospheric pressure, unlike some other coating methods.

Non-limiting examples of biocompatible material combinations that can generate medical implants including a composite coating, as described herein, include: bioactive polymers and bioglass; bioactive polymers and bioinert ceramics; bioactive polymers and bioactive bioceramics; bioceramics and biologically active molecules or compounds; bioactive polymers, bioactive bioceramics and biologically active molecules and compounds; and the like, and combinations thereof. In some embodiments, a composite coating includes a bioactive polymer and a bioceramic. In certain embodiments, a composite coating includes a bioactive polymer, a bioceramic and a biologically active molecule or compound.

Medical implants (e.g., solid materials, support materials, solid support materials) coated with biocompatible composite coatings and biologically active compounds sometimes offer the benefit of reduced recovery time associated with certain medical or dental procedures, when compared to the use of non-treated and/or non-coated implants. Medical implants can be coated with coating embodiments described herein using a method that includes: contacting a support material with a bioactive polymer under a first set of electrophoresis conditions that include applying an electric current of less than 5 mA/cm², thereby depositing the bioactive polymer onto the support material; contacting the support material with bioceramic particles under a second set of electrophoresis conditions, thereby depositing the bioceramic particles onto the support material; and alternating the depositing of the bioactive polymer and the bioceramic particles onto the support material in a predetermined number of cycles, thereby preparing a coated support material, in some embodiments. In certain embodiments, the method further includes contacting the coated support material with cells under cell association conditions. In some embodiments, the method further includes implanting the coated support material into a subject.

A coated solid material, including a support material that includes an exterior surface and a coating adhered to the exterior surface that includes a bioactive polymer and bioceramic particles, where the coated solid material is prepared by a method that includes: contacting the support material with the bioactive polymer under a first set of electrophoresis conditions that include applying an electric current of less than 5 mA/cm², thereby depositing the bioactive polymer onto the support material; contacting the support material with the bioceramic particles under a second set of electrophoresis conditions, thereby depositing the bioceramic particles onto the support material; and alternating the depositing of the bioactive polymer and the bioceramic particles onto the support material in a predetermined number of cycles, thereby preparing a coated support material including on the exterior surface a coating including the bioactive material and the bioceramic material, in certain embodiments. In some embodiments, the coated solid material further includes cells adhered to the coated support material.

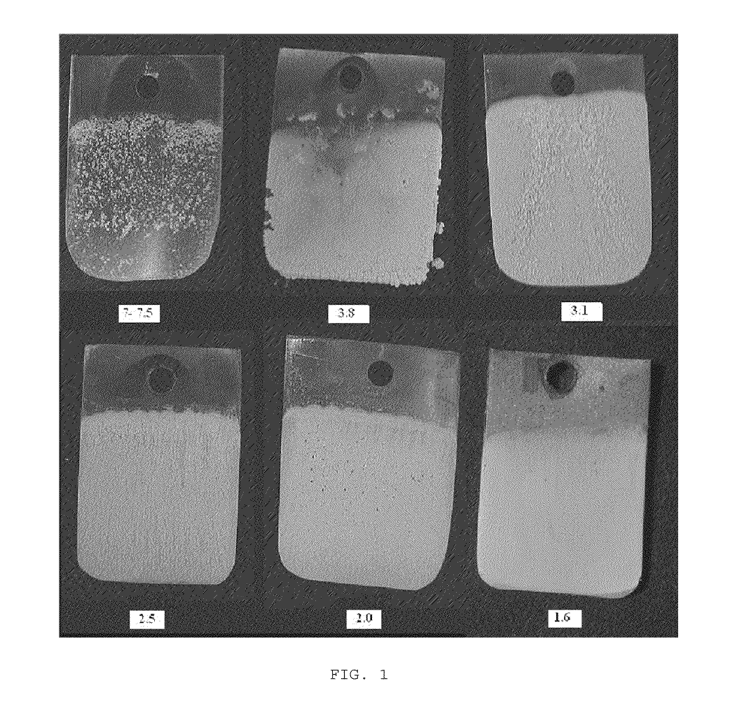

The quantity of material and uniformity of material deposited during electrophorectic deposition sometimes is dependent on one or more of the following parameters: pH of electrophorectic solutions, applied charge density, distance between anode and cathode and duration of deposition. Parameter optimization can be carried out as described in the examples and presented herein. Individual deposition apparatus sometimes vary and determining the optimal parameters for different combinations of support material surfaces and materials for deposition may entail a routine level of experimentation. Any suitable pH can be used for electrophoretic deposition. A pH of about 1.6 can be chosen as an optimum pH for deposition of the composite coatings (e.g., results of studies presented in FIG. 1). FIG. 2 illustrates examples of studies that can be performed to optimize charge density for deposition.

In some embodiments, electrophoresis, under the first electrophoresis conditions, is carried out for less than about 5 minutes (e.g., about 10 seconds, about.30 seconds, about 1 minute, about 90 seconds, about 2 minutes, about 3 minutes, about 4 minutes, about 270 seconds). In certain embodiments, electrophoresis is carried out for less then about 3 minutes (e.g., about 10 seconds, about 30 seconds, about 1 minute, about 90 seconds, about 2 minutes, about 150 seconds). In some embodiments, electrophoresis is carried out for between about 30 seconds and about 90 seconds. In certain embodiments, electrophoresis under the first set of electrophoresis conditions, is carried out at a constant current. In some embodiments, the current is less than about 4 mA/cm². In certain embodiments, the current is less than about 3 mA/cm². In some embodiments, the current is less than about 2 mA/cm². In some embodiments, the current is about 1 mA/cm².

In certain embodiments, electrophoresis under the second electrophoresis conditions is carried out for over 5 minutes (e.g., about 330 seconds, 6 minutes, 390 seconds, 7 minutes, 450 seconds, 8 minutes, 510 seconds 9 minutes, 570 seconds, 10 minutes). In some embodiments, electrophoresis is carried out for between 6 and 8 minutes (e.g., about 6 minutes, 390 seconds, 7 minutes, 450 seconds, 8 minutes). In certain embodiments, the electrophoresis is carried out for more than 10 minutes. In certain embodiments, electrophoresis under the second set of electrophoresis conditions, is carried out at a constant current. In some embodiments, the current is less than about 4 mA/cm². In certain embodiments, the current is between about 2 mA/cm² and about 4 mA/cm². In some embodiments, the current is about 3 mA/cm².

In some embodiments, the bioactive polymer and bioceramic particles are deposited in alternating applications, which can result in alternating layers of the materials. A layer sometimes includes a uniform deposition of a single biocompatible material (e.g., a bioactive polymer, or a bioceramic, or a biologically active molecule or compound). Layers including a single material (layer A, for example) and can be alternated with other layers including a single material, where the other layers are made of a similar or different material (e.g., layer A, layer B, layer C), in some embodiments. In certain embodiments, a layer (e.g., layer A) can be deposited an independent number of times with respect to other layers (e.g., layer B, layer C).

In some embodiments, materials can be alternately applied in cycles. Each unit application or unit of applications can be applied in a cycle, and a cycle often is repeated for the preparation of a coated support material. In some embodiments, a material unit includes one or more bioactive materials applied as independent uniform coatings and a cycle can refer to application of that material unit. In some embodiments, a cycle can refer to application of a single material type. In certain embodiments a cycle can refer to application of two or more material types. Non-limiting examples of composite coatings deposited in a cycle include [layer A]ₙ, [layer B]ₙ, ([layerA][layerB])ₙ, ([layerB][layerA])ₙ, ([layerA]ₓ[layer B]_{y})ₙ, ([layerB]ₓ[layer A]_{y})ₙ, ([layerA]ₓ[layerB]_{y}[layerC]_{z})ₙ, ([layerB]ₓ[layerA]_{y}[layerC]_{z})ₙ, ([layerA]ₓ[layerC]_{y}[layerB]_{z})ₙ, ([layerC]ₓ[layerB]_{y}[layerA]_{z})ₙ, other like combinations, and combinations thereof. In the foregoing embodiments, each of n, x, y and z independently is an integer between about 1 and about 2,000 (e.g., about 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900). In some embodiments, a cycle is repeated about 1 to about 2,000 times.

In certain embodiments, the coating includes layers and a layer includes independent uniform depositions of two or more biocompatible materials (e.g., [layerA_layerB], [layerA_layerC], [layerA_layerB_layerC]). Layers including independent uniform depositions of two or more biocompatible materials can be alternated with other layers, in certain embodiments. In some embodiments, the other layers are made of a similar or different material. In certain embodiments, a layer can be deposited an independent number of times with respect to other layers. In some embodiments, the other layers include a uniform deposition of a single biocompatible material, and in certain embodiments, the other layers include independent uniform depositions of two or more biocompatible materials. In some embodiments, the layers can be alternated in cycles. Non-limiting examples of composite coatings, including layers including independent uniform deposition of two or more biocompatible materials alternate with other layers include coatings of the form [layerA_layerA]ₙ, ([layerA_layerB]_[layerA_layerB]), ([layerB_layerA]_[layerC]), ([layerA_layerB]_{n—}[layerA_layerB]ₓ), ([layerA_layerC]_[layerB_layerB_layerA]), other like combinations, and combinations thereof. In some embodiments, the coating includes layers and the outermost layer is bioactive polymer layer.

In some embodiments, the coating includes layers and a layer includes a non-uniform deposition of two or more biocompatible materials (e.g., [layerAB], [layerBC], [layerBC]_[layerAB]). In certain embodiments, deposition of the two or more materials is simultaneous, and in some embodiments, deposition of the two or more materials is at different times. Layers including non-uniform deposition of two or more compatible materials can be alternated with other layers, in certain embodiments. In some embodiments, the other layers are made of a similar or different material (e.g., [layerAB]_[layerAB], [layerAB]_[layerBC]). In certain embodiments, a layer can be deposited an independent number of times with respect to other layers. In some embodiments, the other layers include a uniform deposition of a single biocompatible material, and in certain embodiments, the other layers include independent uniform depositions of two or more biocompatible materials. In certain embodiments, the other layers include non-uniform deposition of two or more biocompatible materials. In some embodiments, the layers can be alternated in cycles. Non-limiting examples of composite coatings including layers including non-uniform deposition of two or more biocompatible materials alternated with other layers include coatings of the form ([layerAB]_[layerA]), ([layerAB}_[layerAB]), ([layerABC]_[layerA]), ([layerBA]_([layerC]_[layerA])), the like, other combinations and combinations thereof.

In certain embodiments, the coating includes layers that have a thickness of about 0.1 micrometers to about 100 micrometers (e.g., about 0.1 micrometers, about 0.2 micrometers, about 0.3 micrometers, about 0.4 micrometers, about 0.5 micrometers, about 0.6 micrometers, about 0.7 micrometers, about 0.8 micrometers, about 0.9 micrometers, about 1 micrometer, about 2 micrometers, about 3 micrometers, about 4 micrometers, about 5 micrometers, about 6 micrometers, about 7 micrometers, about 8 micrometers, about 9 micrometers, about 10 micrometers, about 12 micrometers, about 14 micrometers, about 16 micrometers, about 18 micrometers, about 20 micrometers, about 22 micrometers, about 24 micrometers, about 26 micrometers, about 28 micrometers, about 30 micrometers, about 33 micrometers, about 36 micrometers, about 39 micrometers, about 42 micrometers, about 45 micrometers, about 50 micrometers, about 55 micrometers, about 60 micrometers, about 65 micrometers, about 70 micrometers, about 75 micrometers, about 80 micrometers, about 85 micrometers, about 90 micrometers, about 95 micrometers, and about 100 micrometers).

In some embodiments, the overall thickness of the coating is about 5 micrometers to about 500 micrometers (e.g., about 5 micrometers, about 6 micrometers, about 7 micrometers, about 8 micrometers, about 9 micrometers, about 10 micrometers, about 12 micrometers, about 14 micrometers, about 16 micrometers, about 18 micrometers, about 20 micrometers, about 22 micrometers, about 24 micrometers, about 26 micrometers, about 28 micrometers, about 30 micrometers, about 33 micrometers, about 36 micrometers, about 39 micrometers, about 42 micrometers, about 45 micrometers, about 50 micrometers, about 55 micrometers, about 60 micrometers, about 65 micrometers, about 70 micrometers, about 75 micrometers, about 80 micrometers, about 85 micrometers, about 90 micrometers, about 95 micrometers, about 100 micrometers, about 125 micrometers, about 150 micrometers, about 175 micrometers, about 200 micrometers, about 225 micrometers, about 250 micrometers, about 275 micrometers, about 300 micrometers, about 325 micrometers, about 350 micrometers, about 375 micrometers, about 400 micrometers, about 425 micrometers, about 450 micrometers, about 475 micrometers, and about 500 micrometers).

Adhesion and Flexibility Measurements

Composite coatings as described herein frequently exhibit good adherence and a low Young's modulus. The coating adhesion rating (e.g., often measured on a scale of 0 to 5, with 0 representing poor adhesion and 5 representing good adhesion) is a measure of the degree of bonding between the composite coating and the support material exterior surface. Adhesion measurements can be performed using tape test ASTM D 3359-97. To test adhesion using this method, a cut is made in the coating, the tape is applied and then removed. The tape and coated surface are inspected to determine if any coating adhered to the tape. Measurement of the amount of coating removed is also recorded. Details of such adhesion measurements are presented in the Examples hereafter.

Young's modulus measures the flexibility of the composite coating on the support material exterior surface. Low Young's modulus values indicate a coating with sufficient flexibility to reduce cracking, chipping and flaking of the coating. Details of Young's modulus measurements are presented in the Examples hereafter, and in FIG. 5.

Biologically Active Molecules and Compounds

In certain embodiments, coated support materials (e.g., coated medical implants) include additional biologically active additives incorporated into or deposited on the composite coatings. In some embodiments the biologically active additives include biologically active molecules and/or compounds. Non-limiting examples of biologically active molecules and compounds include antibiotics, bionutrients, growth factors, hormones, gene regulators, drugs, and the like, and combinations thereof. Coated medical implant embodiments, including biologically active additives may contribute to a reduction in recovery time associated with certain medical or dental procedures when compared to untreated and/or uncoated medical implants.

In some embodiments, a biologically active additive is associated with a coated medical implant. In certain embodiments, a method for preparing a coated support material includes contacting the coated support material with a biologically active additive under deposition conditions, thereby depositing the biologically active additive onto the coated support material. In some embodiments, the biologically active additive is associated by deposition on a composite coating. In certain embodiments, the biologically active additive is associated by incorporation into a composite coating. In some embodiments, the biologically active additive is incorporated into and deposited on a composite coating. In certain embodiments, certain materials are incorporated into a coating in a predetermined number of cycles, and then a biologically active additive is deposited in another set of one or more cycles. The latter approach can be repeated one or more times, and can be supplemented with another set of one or more cycles in which materials other than the biologically active additive are deposited.

Any suitable method for associating biologically active molecules or compounds with coated support materials described herein, can be used. In some embodiments, the biologically active additive is associated with a coated medical implant by electrophorectic deposition. In certain embodiments, the biologically active additive is associated with a coated medical implant by spraying or dipping the implant. In some embodiments, the biologically active additive is associated with a coated medical implant by injecting or infusing the additive into or onto the implant. In certain embodiments, the biologically active additive is two or more biologically active additives. In some embodiments, biologically active molecules or compounds are associated with coated support materials by covalent bonds. In certain embodiments, biologically active molecules or compounds are associated with coated support materials by non-covalent interactions (e.g., biotin, avidin, streptavidin, antibody, antibody fragment). In some embodiments, the biologically active additive is added as a last step in manufacturing the coated support material.

Cells in Association with Coated Solid Supports

Coated solid materials including solid supports and/or medical implants, as described herein, sometimes are associated with cells. In some embodiments, a coated solid material, as described herein, is associated with and/or adhered to cells and/or tissue using a method including: contacting a coated solid material with cells under cell association conditions, whereby the cells adhere to the coated solid material; where: the coated solid material includes a support material having an exterior surface and a coating adhered to the exterior surface, the coating includes a polysaccharide, and the coating includes bioceramic particles. In certain embodiments, a coated medical implant, as described herein, is associated with and/or adhered to cells and/or tissue using a method including: inserting a coated implant into a subject; whereby the coated implant is fused with vasculature in the subject after a period of time; where: the coated implant includes a support material having an exterior surface and a coating adhered to the exterior surface, the coating includes a polysaccharide and the coating includes bioceramic particles.

In certain embodiments, the polysaccharide includes glucose. In some embodiments, the polysaccharide includes a 2-amino-2-D-glucose polymer. In certain embodiments, the polysaccharide includes chitosan. In some embodiments, bioceramic particles include apatite. In certain embodiments, bioceramic particles include wollastonite. In some embodiments, bioceramic particles include apatite and wollastonite. In certain embodiments, bioceramic particles include apatite and wollastonite.

Medical implants prepared using methods as described herein can be associated with cells or tissue from any suitable subject or organism. Non-limiting examples of cells or tissue suitable for association with coated implants prepared as described herein include, fibroblasts (e.g., cornea, tendon, bone marrow, connective tissue), osteoblasts, chondrocytes, odontoblasts, epithelial cells (e.g., absorptive cells, ciliated cells, secretory cells), hormone secreting cells, adipose tissue or cells, neuronal cells, glial cells, muscle cells (e.g., cardiac muscle, skeletal muscle, smooth muscle), and blood cells (e.g., macrophages, neutrophils, lympochytes, leucocytes, erythrocytes), immune cells (T-cells, B-cells, granulocytes), and the like. In some embodiments, the subject or organism is an animal. In certain embodiments, the subject or organism is a mammal. In some embodiments, the subject or organism is a primate. In some embodiments, the subject or organism is a human. In some embodiments the cells are mammalian cells. In certain embodiments, the cells are human cells.

In some embodiments the cells are in a vasculature structure. Vascular structures often are a body part or structure that is composed of or provided with blood vessels. Coated implants prepared as described herein sometimes are associated with cells that form part of the arrangement or distribution of blood vessels in various organs and/or body parts (e.g., bones, lungs, brain, heart, blood vessels, the like, and combinations thereof, for example). In some embodiments the cells are derived from and/or form vascular structures. In some embodiments, the cells are derived from and/or form bone. In certain embodiments, the cells are derived from and/or form cartilage. In some embodiments, the cells are derived from and/or form muscle. In certain embodiments, the cells are derived from and/or form connective tissue.

The coated support materials described herein can be associated with cells using a variety of methods. In some embodiments, the cells are associated with coated solid supports or coated medical implants under cell association conditions. In certain embodiments, the cells are associated with coated support materials or coated medical implants in vitro, utilizing in vitro cell association conditions (e.g., cell culture conditions specific for a particular cell type, cell association conditions for a particular cell type, the like, and combinations thereof). In some embodiments, in vitro cell association conditions include one or more of media (e.g., cell culture media, simulated body fluids), nutrients, biologically active molecules or compounds, matrix material and/or biologically compatible composite coating for cell viability. In certain embodiments, in vitro association conditions include simulated body fluids. In some embodiments, the cells are associated with coated solid supports or coated medical implants, utilizing in vivo cell association conditions (e.g., implanted in subject). In certain embodiments, coated support materials or coated medical implants are implanted into a subject.

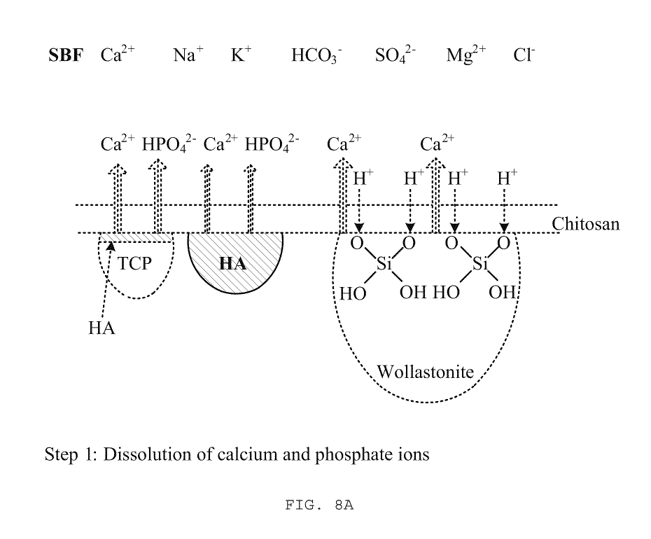

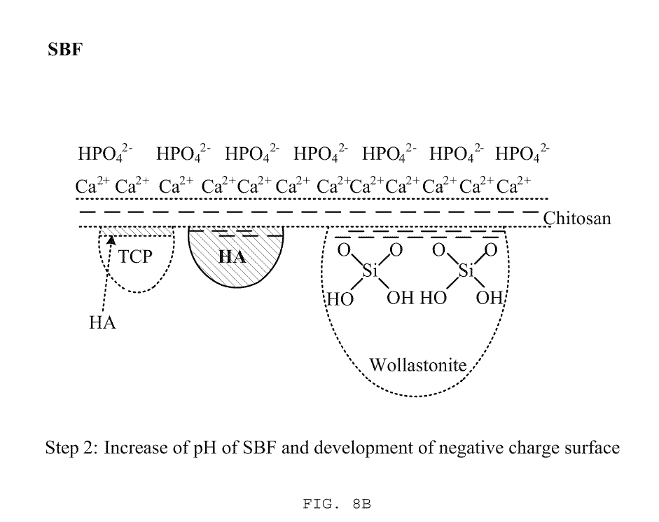

Cell association with coated medical implants can be observed and/or measured using standard detection and visualization methods (e.g., observation of bone callus formation using the naked eye, radiographic visualization (e.g., X-ray) microscopic visualization, detection of incorporated radio-isotopes, fluorescence imaging, and the like, and combinations thereof). Measurements of the number and/or types of cells associated with coated medical implants can be compared to observations made of cells associated with uncoated implants, to determine the efficacy of treatment with the coated implant. Another measure useful for determining the efficacy of treatment with coated implants is identification of the timing of cell proliferation and cell type appearance. Coated implants often reduce the time of appearance of certain cell types and sometimes promote cell proliferation at an earlier time than uncoated implants. The reduction in time for cell proliferation, cell adhesion and appearance of certain cell types may contribute to the overall reduction in recovery time sometimes associated with treatment using support materials or medical implants coated with biocompatible composite coatings. Measurements of cell association are described further in the examples and are illustrated in FIGS. 10A-16D. An example of radio-isotope incorporation for coated and uncoated implants inserted into subjects is given in Table 1, presented in the Examples hereafter.

Association of Tissue

Association of tissue (e.g., vasculature) with medical implants inserted into a subject can be used to determine the success or efficacy of treatment with the inserted implant. Any tissue can be associated with an implant, including without limitation, connective tissue, tissue of skin, heart, lung, vein, artery, blood, brain, muscle, and the like. Vascularization involves the formation of new blood vessels, or microvascular networks that provide blood and nutrient flow to newly developing tissue at or near the site of implant insertion. Insertion of a medical implant often involves a surgical procedure that may disrupt vascular structures at or near a region where an implant is inserted. As noted above, when implants manufactured from uncoated bioinert materials are implanted into a host, a zone of tissue avoidance or tissue rejection sometimes occurs in the vicinity of the inserted implant, thereby slowing the reassociation of cells and vasculature with the damaged tissue.

Implantation Procedures

Implantation of coated support material as described herein can be performed using any suitable implantation method. In some embodiments, medical implants described herein can be inserted using implantation methods involving invasive surgery, and in certain embodiments, implantation procedures can be performed using arthroscopic surgery. Orthopedic surgeries can be used to insert coated medical implants prepared as described herein to correct conditions involving the musculoskeletal system or a subject. Non-limiting examples of orthopedic surgeries to correct conditions involving the musculoskeletal system include: joint (e.g., hip, knee, elbow, shoulder) repair or replacement; bone repair (e.g., plates, pins, screws, and the like); bone reconstruction or grafting; muscle and/or connective tissue (e.g., tendons and ligaments) repair and/or reattachment; and the like, and combinations thereof. Dental surgeries (also referred to as prosthodontics or prosthetic dentistry) and/or maxillofacial surgeries can be used to insert coated medical implants prepared as described herein to correct conditions involving teeth, oral and/or maxillofacial tissues. Non-limiting examples of dental and/or maxillofacial surgeries to correct conditions involving teeth, oral and/or maxillofacial tissues include: tooth repair and/or replacement (e.g., crowns, bridges, dentures, and the like); mandibular repair or replacement; insertion of maxillofacial prosthetics (e.g., artificial eyes, and other facial prostheses).

Subjects

Any organism in need of a medical implant (e.g., for musculoskeletal, maxillofacial, dental and mandibular repair or replacement) is suitable as a subject for implantation of a coated support material. Non-limiting examples of subjects that may benefit from procedures involving implantation of a coated support material include humans, primates, canine, equine, porcine, bovine, and more generally any animal suffering from a condition treatable by a coated device as described herein. Non-limiting conditions and procedures include joint (e.g., hip, knee, elbow, shoulder) repair or replacement; bone repair (e.g., plates, pins, screws, and the like); bone reconstruction or grafting; muscle and/or connective tissue (e.g., tendons and ligaments) repair and/or reattachment; tooth repair and/or replacement (e.g., crowns, bridges, dentures, and the like); mandibular repair or replacement; insertion of maxillofacial prosthetics (e.g., artificial eyes, and other facial prostheses), and the like, and combinations thereof).

Assessment of Neovascularization Progress

Successful implantation of coated implants described herein includes osseointegration of the implant into the tissue and vascularization of the implant site and tissue around the implant site. Failure of successful implantation is readily identified. Failure to successfully integrate often results in zones of tissue avoidance and rejection around the implant. Tissue necrosis sometimes is also visible as a result of failure of implantation. Tissue avoidance and rejection around the implant sometimes is seen using bioinert materials.

Successful osseointegration can be observed by the formation of new bone growth or callus in the bone in which the implant is inserted, in some embodiments. Osseointegration refers to the process of bone growing right up to the implant surface. In some embodiments, a limited amount of soft tissue, a minimal amount of soft tissue, a reduced amount of soft tissue, or no soft tissue, connects the bone to the surface of the implant. In certain embodiments, a limited amount of scar tissue, cartilage or ligament fibers, a minimal amount of scar tissue, cartilage or ligament fibers, a reduced amount of scar tissue, cartilage or ligament fibers, or no scar tissue cartilage or ligament fibers are present between the bone and implant surface. The direct contact of bone and implant surface can be verified microscopically. When osseointegration occurs, the implant is tightly held in place by the bone. In certain embodiments, cells and tissue will visibly adhere to the coated surface of the implant.

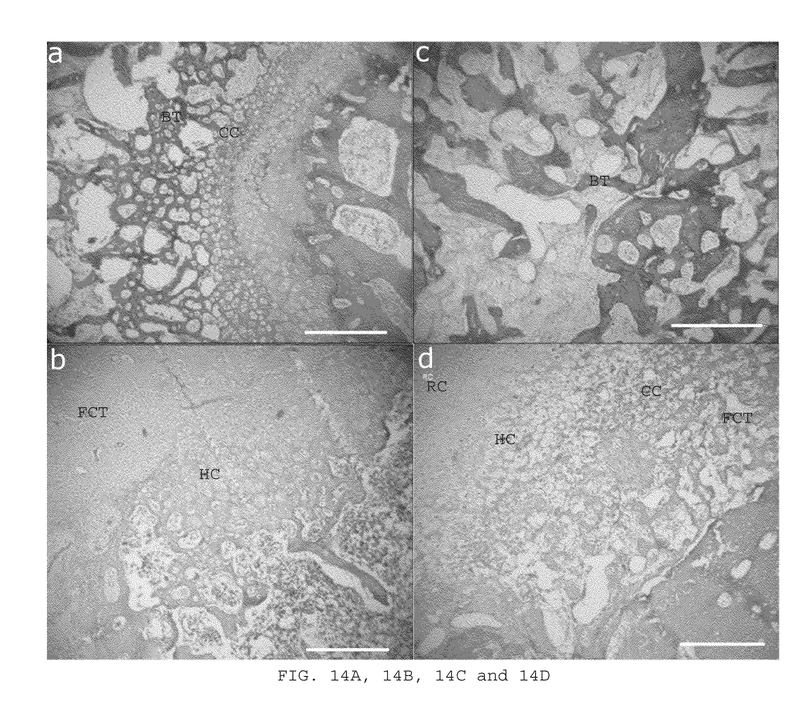

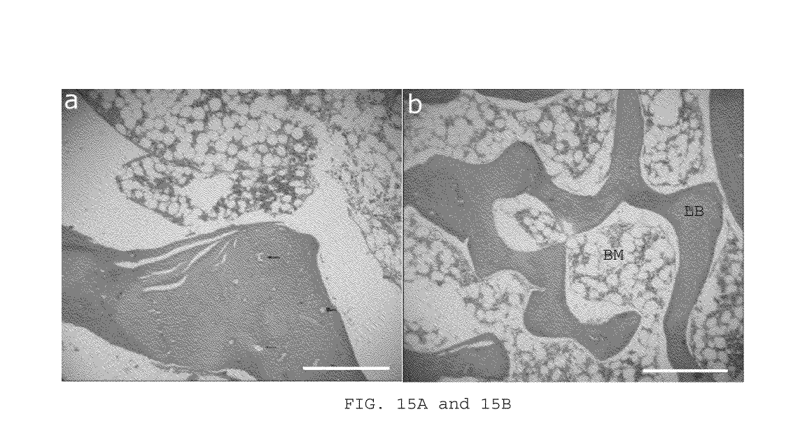

Additional methods, both direct and indirect, can be used to assess the progress of successful implant vascularization. Fluorescence labeling (see. FIGS. 14A-14D) can be performed to identify mineralization regions (e.g., calcification of cartilage). Bone scintigraphy can also be performed (see Table 1) to determine the rate of nutrient uptake over the course of implantation. Typically, incorporation of radio-isotope will initially increase as healing progresses followed by a decrease when healing is complete. Fluorescence labeling and bone scintigraphy are indirect measurements of vascularization, as incorporation of calcium or radio-isotope is mediated by delivery of the appropriate molecules to the wound site.

Gross observations can be made for direct assessment of vascularization (e.g., neovascularization or revascularization). Visual observations of the wound site both internally and externally sometimes can provide an indication of the degree of neovascularization. In some embodiments, the implant/bone surface will show fibrous growth (formation of bone callus) and may additionally appear pinkish or red as a result of the formation and adherence of functional microvascular networks, with red blood cell perfusion, to a coated implant. Direct visual assessment of bone healing and vascularization are shown in FIGS. 10A-13D.

### EXAMPLES

The examples set forth below illustrate certain embodiments and do not limit the disclosed technology.

### Example 1

### Chitosan Reinforced Apatite-Wollastonite Coating by Electrophorectic Deposition on Titanium Implants

Conditions for electrophorectic deposition of composite coatings including chitosan and apatite-wollastonite, were investigated for preparation of coated medical implants.

A novel bioactive porous apatite-wollastonite/chitosan composite coating was prepared by electrophoretic deposition. The influence of synthesis parameters like the pH of suspension and current density were studied. X-ray diffraction confirmed the crystalline phase of apatite-wollastonite in powder as well as in the composite coating with coat crystallinity of 65%. Scanning electron microscope imaging showed that the coating showed porosity with interconnections, and good homogeneity between the phases. The addition of chitosan increased the adhesive strength of the composite coating. Young's modulus of the coating was found to be 9.23 GPa. Studies presented herein suggest sheet-like apatite growth on surfaces coated with the composite coating, unlike ball-like growth found in bioceramics coatings. The role of chitosan in apatite growth mechanisms, in simulated body fluid, was studied. In the presence of chitosan, sheet-like evolution of the apatite layer involved dense negatively charged surfaces with homogenous nucleation. The results suggest that incorporation of chitosan with apatite-wollastonite in composite coatings could provide in vitro bioactivity with enhanced mechanical properties.

Titanium and titanium alloys have shown high potential for load bearing in bioimplant applications due to their biocompatibility and reliable mechanical properties. But, from a biochemical point of view, they are considered nearly inert materials.

Materials and Methods

Preparation of materials used in Electrophoretic Deposition

Apatite-wollastonite (AW) powder formed by modified sol-gel route was used in this study for synthesizing the composite coating. Briefly, an ionic solution of calcium nitrate and magnesium nitrate was prepared and mixed with organic solution of Tetraethyl Orthosilicate (TEOS), methanol and calcium fluoride in sol-gel apparatus. All analytical-grade chemicals were used. After the formation of gel, it was calcined at 200 degrees Celsius and then sintered at 950 degrees Celsius in tubular furnace (hcs-Tub, hecons, Mumbai, India). The powder obtained was ball milled at 150 rpm for 2 hours. Particle sizing was carried out using a dynamic light scattering (BI-9000 AT Digital Autocorrelator, Brookhaven Instrument, USA) and was found to be 200 nm. Chitosan was obtained from Otto chemicals (98% Deacetylated). Titanium sheet (Manhar metal suppliers, Mumbai, India) of dimension (10 mm×15 mm×0.5 mm) was used as the test substrate. The substrates were etched with 2% hydrofluoric acid (HF) for 1 minute, then rinsed with MilliQ water (Millipore) and air-dried before use.

Deposition Details

Titanium (Ti) test samples were used as both anode and cathode. Distance between the electrodes was maintained at 10 mm. The ceramic particles of apatite-wollastonite were dispersed ultrasonically in ethanol for 30 minutes at 20 Hz (98 kW) in an ultrasonic vibrator. Electrophoretic deposition was performed using a suspension of 2 g/L AW particles in ethanol. The pH of the ceramic suspension was optimized after carrying out repeated experiments and was fixed at pH of 1.6. A suspension of 0.2% of chitosan was prepared in 2% acetic acid solution. Cathodic deposition was performed on Titanium (Ti) sheet with a coating area of roughly 10 mm×10 mm. A current density of 3 mA/cm² was selected to coat ceramic and 1 mA/cm² to coat chitosan. A repeated deposition method was applied to reduce formation of cracks in the coating. To start with, surface of titanium was coated with thin layer of chitosan followed by three alternate coating cycles of ceramic and chitosan to obtain homogenous composite coating. The last coat of chitosan was repeated two times so as to encapsulate composite coating by polymer thereby preventing the erosion of the final composite coating. Representative images of the coatings are shown in FIGS. 1, 2 and 4A-C.

Characterization of Composite Coatings

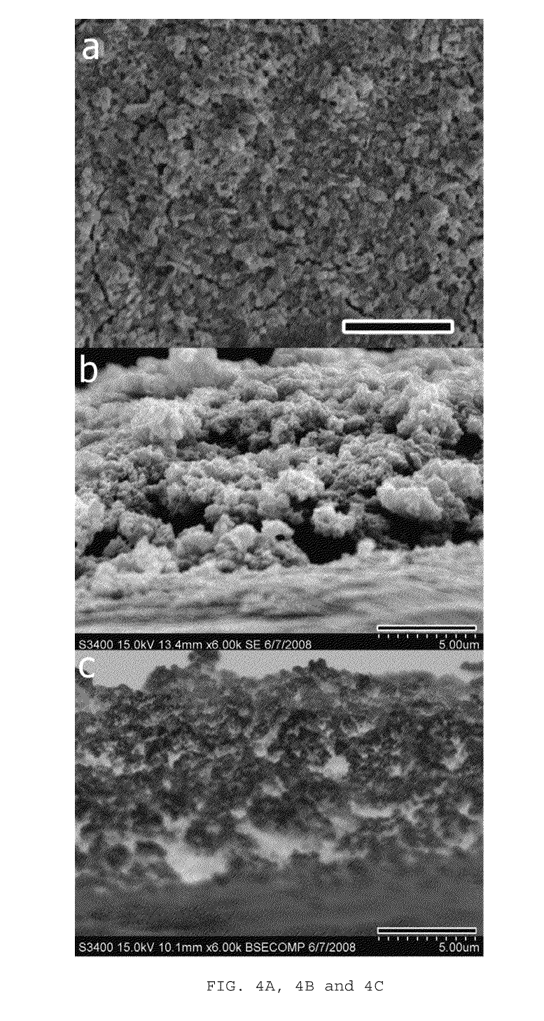

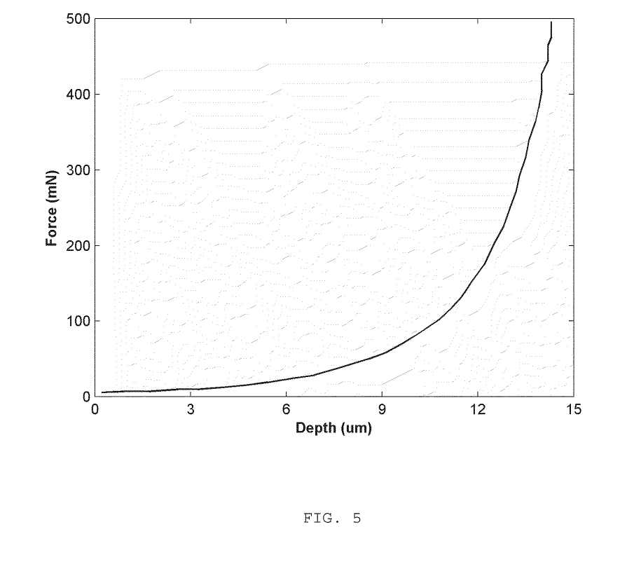

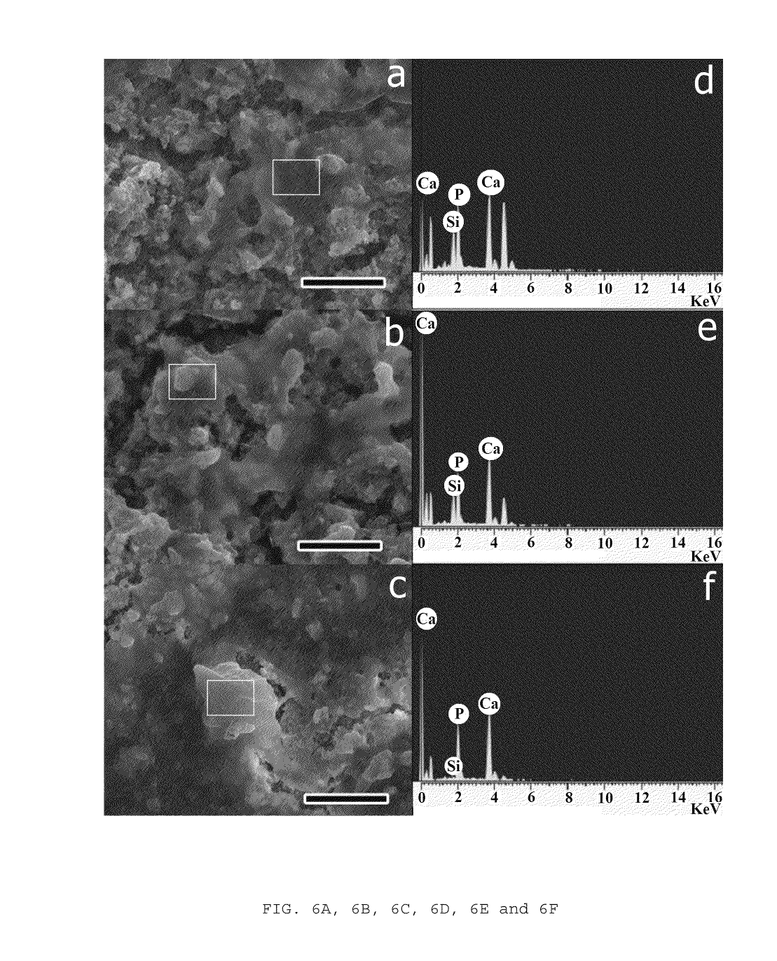

Compositional phase analyses and crystallinity were determined using X-Ray Diffraction (XRD: X'Pert PANalytical, Philips, see FIG. 3). The operating conditions were 40 kV and 30 mA by using Cu Kα monochromatic radiation with a step size of 0.2°/15 seconds. Scanning Electron Microscopy (SEM, JSM-6400, JEOL, Japan) and energy dispersive X-ray spectroscopy (EDAX) were employed to investigate the morphological features and elemental composition of the coatings. The working distance and voltage used during the scanning electron microscopy was 15 mm and 20 kV respectively. Micro-Raman investigations were performed on localized areas (illustrated in FIGS. 6A-6F) of the coated sample using Raman spectrometer (LabRAM HR800, Jobin Yvon, France). An Argon (Ar⁺ 514-nm) laser source with an intensity approximately equivalent to 10 milliwatts (mW) was used.

Mechanical testing of Composite Coatings—Tape test

For assessing the adhesion of the composite coating on titanium substrate, a standard test method (Tape test—ASTM D 3359-97) was used. This was measured by applying a pressure-sensitive tape (EURO Tape, Century distributors (P) Ltd.) on the composite coating. Coverage of coated substrate was quantified using Matlab (version 7.1).

Mechanical testing of Composite Coatings—Nanoindentation

Young's modulus of composite coating was measured using indenter type Berkovich B3 (Universal Nanomechanical Tester UNAT, ASMEC). Coated sample was measured 15 times at 3 different forces of 50, 150 and 500 mN each. Young's modulus was inferred from the load-displacement curve for the composite coating. A representative load-displacement curve is shown in FIG. 5.

Hemolysis Assay

The hemocompatibility of coated substrate was evaluated using methods known. Erythrocytes in normal saline served as a negative control while erythrocytes in distilled water served as a positive control. Percentage Hemolysis was then calculated according to Formula 1.

$$\% Hemolysis = \frac{Absorbance}{Absorbance 100 \% Hemolysis} \times 100 \qquad ( 1 )$$

Bioactivity Studies

The titanium substrates were soaked in standard simulated body fluid (SBF) which is similar in ionic concentration to human plasma. The concentration of the ions was (mmol/dm³) Na⁺ 142.0, K⁺ 5.0, Mg²⁺ 1.5, Ca²⁺ 2.5, Cl⁻ 147.8, HCO₃₋ 4.2, HPO₄²⁻ 1.0, SO₄²⁻ 0.5. The samples were immersed in SBF for 7 days, 14 days and 21 days. All samples were incubated at 37° C. Following incubation, each sample was taken out and examined for the deposition of hydroxyapatite. The coatings were sputter coated with gold alloy. Apatite growth was identified using SEM-EDAX and Micro-Raman spectroscopy.

Results

pH Optimization

The stability of the suspension sometimes plays a role in achieving suitable electrophoretically deposited coatings. When the pH of the ceramic suspension in ethanol was 7.5; there was very little deposition of ceramic particles on the cathode, possibly due to the ceramic particles having a lower positive charge density. At a low pH of 1, even though the positive charge density on ceramic particles was high, observed ceramic deposition was low, possibly due to an increased ionic concentration, which led to a drop-down of the applied voltage. Thus, pH optimization was performed using 1N HCl over a range of pH 1-7.5 at fixed current density of 3 mA/cm² and coating duration of 1 min. Quality of the coating was determined by the absence of spalling, pitting and cracks on the coating. As shown in FIG. 1, it was found that at pH 1.6, the ceramic coating was uniform, dense and without any major surface defects.

Current Density Optimization

Current density affects the kinetics of EPD process. Higher current density has the benefit of faster deposition kinetics but at the cost of surface non-homogeneity. Lower current density has slower deposition kinetics but the expected coating is dense, homogeneous and more uniform. Therefore, the ceramic was coated using EPD apparatus at different current densities ranging from 3-9 mA/cm² and optimized in order to get a uniform coating at a faster rate.

At 7 and 9 mA/cm² cracks appeared in the coating in the beginning of the process only. 5 mA/cm² produced a uniform coating initially but later on caused spalling. FIG. 2 shows that a current density of 3 mA/cm² is best suited for producing thick depositions without any deformation.

Phase Analysis and Morphological Studies

The XRD patterns of AW powder and composite coating are shown in FIG. 3. XRD studies show the presence of different phases; wollastonite (W, JCPDS 72-2284), hydroxyapatite (H, JCPDS 09-0432) and TCP (T, 03-0713) in powder and coated sample. Ceramic powder diffraction pattern indicates presence of more crystalline phases with strong diffraction plane (120) of wollastonite and (211) of HA. In the coated sample, the Bragg angles of different phases are the same as seen in the diffraction patter of the ceramic powder. Also, the ratio of the intensities of powder and coated sample are almost the same, indicating that there is no preferred orientation of crystallization during the coating process. There is an added amorphous band in coated sample which may be due to the amorphous nature of chitosan. The crystallinity of the apatite-wollastonite powder and the composite coating was calculated from the XRD pattern and was found to be 93% and 65%, respectively.

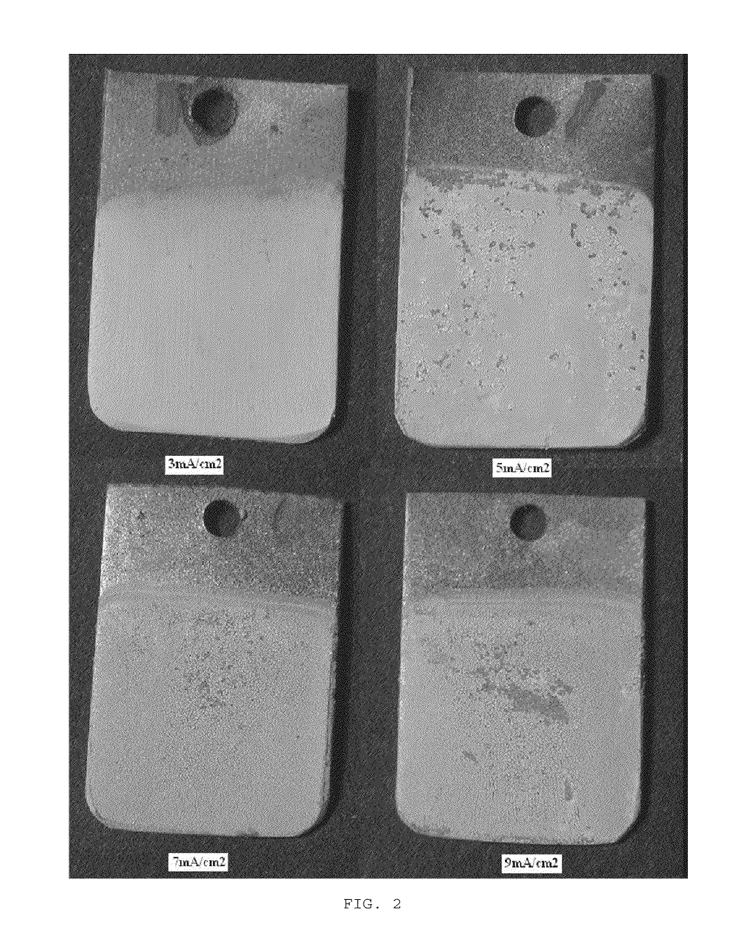

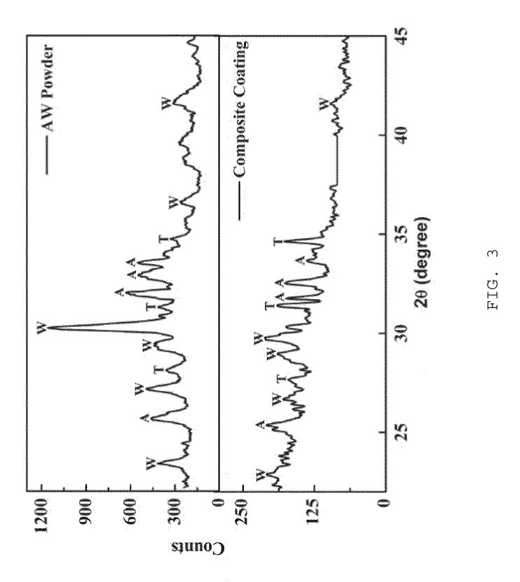

Scanning electron microscopy was employed to investigate the morphological features of the coated sample. It can be inferred from the micrographs (see FIG. 4A) that the composite coating has a porous structure and particles are fused together to give an irregular morphology. The coating has interconnected pores and the thickness of the coat is found to be approximately 10 μm (FIG. 4B). FIG. 4C illustrates a back scatter electron micrograph image with compositional contrast (e.g., BSE COMPO) of a cross section of a coating, the darker and lighter patches correspond to chitosan and AW ceramic particles, respectively, and good homogeneity is seen between the two phases.

Mechanical Strength Testing—Tape Test

The adhesion of the composite coating on titanium substrate was measured using standard test method (Tape test—D 3359-97). This was done by applying a pressure-sensitive tape (EURO Tape) over cuts made on the coating. In the ceramic coating, the coated area removed was found to be 66% and in the composite coating, the coated area removed was found to be 21%. Classification of adhesion test were evaluated using a scale from 0 to 5 i.e., 0 corresponds to very poor and 5 to very good adhesion respectively. Classification of the coating was done using the standard chart provided with the pressure sensitive tape. With polymer, the coating had an adhesion rating of 2B and without polymer the coating had an adhesion rating of OB.

Mechanical Strength Testing—Nanoindentation

FIG. 5 displays average load-displacement curves from all measurements for a representative coated sample. The figure shows that the indentation depth increases much faster at smaller loads than at higher loads. The average depth value of Young's modulus was found to be 9.23±0.94 GPa. Results from still higher depths may be influenced by the metallic substrate.

Hemocompatibility

It is desirable that a biocompatible implant also is hemocompatible; and especially it should not lead to hemolysis (e.g., lysis of red blood cells (RBCs)). The interaction of the coated sample with RBC's was evaluated as these cells serve as model cell membranes. When the values of % Hemolysis were plotted in a bar graph format, the hemocompatibility of the composite coating was found to be approximately 2%.

Bioactivity

Bioactivity of orthopedic materials is characterized by the capability of forming bone-like apatite in vitro and in vivo. Apatite growth was studied by visualizing different morphological features and elemental composition using SEM-EDAX and confirming phosphate vibrational mode in apatite using Raman spectroscopy. SEM images of composite coating immersed in SBF showed increased apatite growth from 7 to 21 days. Ceramic coated and composite coated samples were soaked in SBF to study their effect on the morphology of apatite growth.

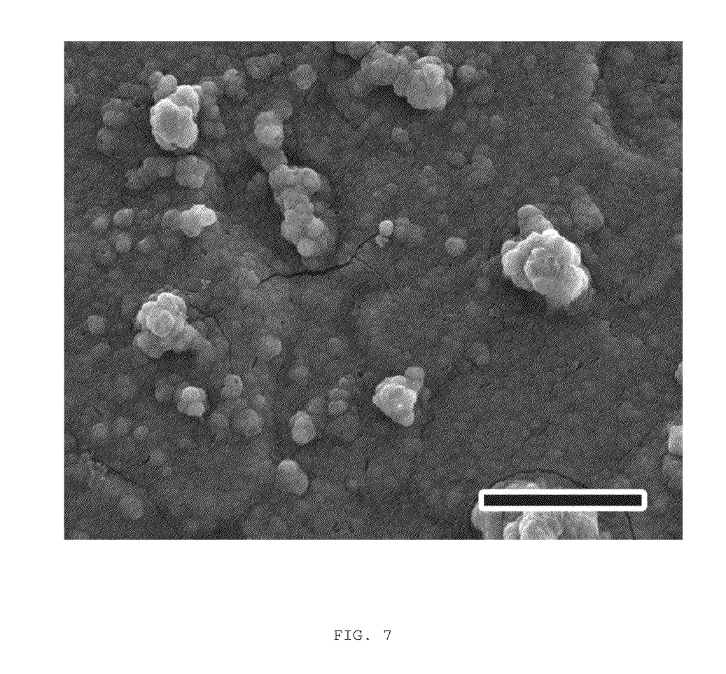

FIG. 6 shows that apatite growth has sheet-like morphology for composite coatings, unlike the ball-like morphology on the AW coated sample, which is commonly observed in bioceramics (see FIG. 7). Apatite precipitation was confirmed for all samples from the calcium/phosphate (Ca/P) ratio of 1.67, as measured by EDAX. From the EDAX analysis, it was found that the silica (Si) concentration was large on day 7 because EDAX was sampling into underlying apatite-wollastonite, and hence the Si peak is large. However, at 14 days and 21 days, SBF samples showed drastic decrease in Si concentration because of the thicker apatite layer which reduced the contribution of underlying apatite-wollastonite layer to the EDAX signal. This gradual decrease in Si content and constant ratio of Ca/P equal to 1.67, indicated apatite was present and it was growing over the composite coating from 7 days to 21 days. FIGS. 8A-8D show a proposed mechanism of apatite growth in the composite coating.

Micro-Raman spectroscopy was used to identify different vibrational modes that are Raman active, before and after immersion in SBF for 7, 14 and 21 days. FIG. 9 shows the Raman spectrum of coated samples in the region between 250-1100 cm⁻¹. Two intense Raman-scattering bands of hydroxyapatite are identified for control sample, which are associated with two normal modes of frequencies of the PO₄⁻³ tetrahedron viz. symmetric stretching of P—O bonds at 1000 cm⁻¹ (v₁ frequency) and asymmetric P—O stretching at 1032 cm⁻¹ (v₃ frequency). The small peak towards left of v₁ frequency of PO₄⁻³ belongs to P—O symmetric stretching in Ca₃(PO₄)₂ phase in composite coating. For the sample immersed in SBF for 7 days, there is an appearance of broad band at 546 cm⁻¹ and a peak at 620 cm⁻¹, which are assigned to breathing vibrations of oxygen atom in Si—O network. The band at 795 cm⁻¹ is associated with symmetric stretching vibrational mode of Si—O. Thereafter, for 14 and 21 days there is a progressive decrease in their intensities.

Discussion

An electrophoretic process for deposition of ceramic and polymer enables deposition of uniform coatings on substrates of complex shapes. It has been shown to be a versatile technology for preparing composite coatings on metallic substrates with controlled homogeneity and surface roughness. It is an efficient technique for depositing heat sensitive biopolymer. Chitosan has the advantage of providing high flexural strength and also it induces osteoconductive properties. By choosing appropriate proportions of chitosan and AW, bioactive coatings with greater mechanical strength can be achieved.

The pH of a suspension can affect the particle charge distribution and ionic conductivity of the suspension, which in turn can affect the electrophoretic mobility of the particles with respect to ions. Thus, dynamics of the EPD process can be controlled by selecting an appropriate pH of the electrophorectic suspension. Electrophoretic velocity of particles can be related to the charge on the particle and the electric field, and can be calculated using Formula 2,

$$v = \frac{EQ}{4 π r η} \qquad ( 2 )$$

where, E is applied electric field, Q is particle charge, r is particle radius and η is viscosity of the suspension. For a given concentration of a suspension, its viscosity is invariant and the only thing that varies with the pH of the suspension is the product EQ. At pH 7.5, there is minimum positive charge deposition on particles and finite electric field, i.e., Q→0 implies v→0, which results in minimum deposition of particles on titanium substrate as is clearly shown in FIG. 1. At pH 1, although the expected positive charge deposition is high, the excess of ionic concentration in the suspension leads to the formation of an electric double layer on electrode and results in an eventual lessening of the electric field, i.e., E→0 implies v→0. Here too, a thin deposition of particles will occur as the electric field is not sufficient for electrophoresis. Hence, there must be an optimum value in between the pH of 1 to 7.5, where the electrophoretic velocity is at a maximum and this has been observed by the uniform dense coat formed by deposition at a pH of 1.6.

Kinetics of EPD process is governed by applied current density on electrode. Current density influences the rate of mass deposition on the cathode and can be calculated using the Hamaker Equation (Formula 3):

m=CviρSt (3)

where, m is mass deposited, v is electrophoretic mobility, i is current density, ρ is resistivity, S is surface area of electrode and t is time duration of coating. There is a linear relation between rate of mass deposition and current density. At low current density (3 mA/cm²), the particles have sufficient time to rearrange resulting in a more uniform coating. As the current density is increased to 7-9 mA/cm², more particles are deposited, faster and with lesser rearrangement, resulting in spelling and cracking in the coat.

XRD of ceramic powder confirms the presence of apatite and wollastonite phases. TCP phase is also present in small proportion. XRD pattern of coating shows decreased intensity of peaks and amorphous band; this might be because of very thin coating. Decrease in crystallinity of composite coating observed may be due to incorporation of chitosan. Also there is no preferred orientation of crystallization in any phase of coating because deposition of multi-phase ceramic particle leads to disorder in crystallization planes.

SEM image of composite coating shows interconnected pores (see FIGS. 4A-4C and 6A-6F). It has been suggested that the interstices and pores in coat are pathways for diffusion of nutrition elements, vascularization and cell growth. The BSE-COMPO image (see FIG. 4C) shows that AW ceramic is embedded inside chitosan layer providing mechanical strength to composite coating. Hence, it can be deduced that EPD is a suitable processing technique to form an interconnected porous microstructure with desired thickness by controlling pH, current (e.g., charge density) and time of deposition.

Total coating strength is a sum of the adhesive force between coat and substrate and the interparticle cohesive force. To increase the adhesive strength of coating, chitosan was employed as first layer of the composite coating. Polymeric layer increases effective contact area of composite coating as compared to ceramic coating alone. Also, within the coating layer, ceramic particles are enwrapped in chitosan layer which provide them bulk strength. The results presented here indicate that chitosan; can be an effective binder, can provide adhesion of the particles to the substrate and can reduce or prevent cracking.

Young's modulus of the coating can be affected by the packing of particles, porosity and composition of materials used in the coating. The presence of chitosan can increase the porosity of the coating, but it also leads to a decrease in Young's modulus due to the flexible nature of chitosan, as compared to the bulk elastic modulus of AW. Young's modulus decreases due to presence of the polymer in AW-polymer composite coating, leading to an increase in the flexural strength of the composite. Thus, the incorporation of chitosan causes a decrease in the Young's modulus, but simultaneously increases the adhesive strength of the composite coating.

Hemolysis study is a significant test for biomaterials. The hemolytic reaction level caused by the toxic materials is generally larger than the toxicity reaction level produced in cell culture. Materials that cause hemolysis often are regarded as toxic. According to the standard, American National Standard Institute/American Dental Association (ANSI/ADA) Specification No. 41 (Biological evaluation of dental materials. Washington D.C., ANSI/ADA, 1979), any material resulting in less than 5% hemolysis is considered hemocompatible. The hemolytic ratio of the composite coating was found to be 2% and thus the coating is hemocompatible.

Investigating the biological behavior of biomaterials in SBF is considered a suitable and efficient method to evaluate the bioactivity of biomaterials in a body-like environment. Based on studies using SBF, a mechanism of apatite growth on AW-Chitosan composite coatings is proposed and the effect of the biopolymer on the morphology of apatite growth is shown. Composite coatings prepared as described herein, have different phases and each phase has a definite role in apatite formation.

Step 1: Dissolution of Calcium and Phosphate Ions

When the coating is immersed in SBF (pH 7.4), ion exchange occurs between the surface layer of the coating and the solution as a result of the different chemical potentials of the ions. The ion exchange in the wollastonite phase is coupled with the dissolution of calcium ions and the absorption of protons leading to formation of silanol groups on the coating surface. TCP gives insoluble hydroxyapatite and releases calcium and phosphate ions upon hydration during immersion in SBF (Formula 4).

4Ca₃(PO₄)₂+H₂O→Ca₁₀(PO₄)₆(OH)₂+2Ca²⁺+2HPO₄²⁻ (4)

As the solubility of hydroxyapatite in water is very low at 37° C., the release of calcium and phosphate ion is less.

Step 2: Increase of pH of SBF and Development of Negative Charge Surface

Release of calcium and phosphate ions from the ceramic surface and simultaneous consumption of protons from the SBF causes an increase in the pH of the medium. Surface silanol group of wollastonite in high pH medium stabilizes to form Si—O— group followed with densification of silica layer, which results in an increased surface charge density. As the isoelectric point of hydroxyapatite is between pH 7-8.5, at higher pH levels, the phosphate and hydroxyl groups present will have more negative charges on the surface. Similarly, the primary hydroxyl group of chitosan also contributes to the negative charge as it forms Ch-O— group at pH levels greater than the isoelectric point (pH 6.4) of chitosan (Ch-OH) (Formula 5).

Ch-OH+OH⁻→Ch-O⁻+H₂O (5)

There is a strong negative charge contribution from the polymeric layer, which in turn provides dense homogeneous nucleation sites for apatite growth.

Step 3: Increase of Ionic Activity Product (IAP)

Increases in the ionic concentrations of calcium ions, phosphate ions and hydroxyl ions in SBF will increase the IAP of apatite and eventually lead to a higher degree of super-saturation in the solution. The negatively charged surface of the composite coating will attract calcium ions onto its surface by electrostatic force. The deposited calcium ions may then attract their counter ions for apatite formation from the SBF.

Step 4: Precipitation of Apatite

Formation of a double layer and increases in the IAP of apatite induces calcium-rich phosphate precipitation on the coating surface. On successive layers of newly formed Ca-P layer, calcium ion deposition will be lesser as compared to the preceding layer. Thus, each successive layer deposited will be of calcium-poor phosphate. As the calcium-poor phosphate has lower solubility, it is more stable and will eventually be converted into crystalline apatite.

Apatite growth was also studied using Raman spectroscopy (see FIG. 9). The increase in intensity of bands at 546, 620 and 795 cm-1 is due to dissolution of calcium ion from wollastonite and formation of silica-rich layer. Thereafter, for 14 and 21 days there is a progressive decrease in their intensities owing to the formation of thick apatite layer onto the composite surface. Broadening of band at 965 cm-1, which corresponds to Ca3(PO4)2 phase, for 7 and 14 days is related to the dissolution of calcium phosphate, which is the first step in apatite growth mechanism. Again emergence of a peak at a slightly lower frequency on the 21st day is due to the fact that crystallized apatite is supported on rich-calcium phosphate layer.

Thus, the sheet-like morphology of apatite growth may involve the densely charged surface of the composite coating, which may be attributed to the presence of high surface charge density of chitosan. It is generally observed that apatite growth on bioglasses and HA attains a ball-like morphology, which is due to presence of grain boundaries. A grain boundary often serves as discontinuity for continuous apatite growth. Chitosan might also be masking the grain boundary effect of ceramic particles leading to sheet-like apatite growth.

Conclusions

A technique for a composite coating of apatite-wollastonite and chitosan on titanium substrates was developed using the ambient temperature and pressure technique of electrophoretic deposition (EPD). The current density and pH of suspension were optimized together to get uniform and crack-free coating. Depositions showing decreased cracking and spelling were obtained using a current density of 3 mA/cm² and pH of 1.6, for example. The XRD results confirmed the presence of apatite, wollastonite and a small amount of TCP phases in both powder and the coating, and furthermore, the coating had reduced crystallinity as compared to AW powder, which may be due to addition of chitosan. The coating had a porous structure with ceramic particles enwrapped inside the polymer layers. Chitosan incorporation enhanced the adhesive strength of the composite coating. Bioactivity studies showed that apatite growth had sheet-like morphology mainly due to the dense homogenous nucleated sites provided by high surface charge density of chitosan. Sheet-like apatite growth is expected to produce homogenous bioactive surface as compared to ball-like apatite. Chitosan reinforcement in composite coating showed good bioactivity and increased mechanical strength. Considering its economic and simple production, apatite-wollastonite-chitosan composite coating seems to be an attractive candidate to improve the performance of metallic implants.

### Example 2

### Bone Healing Performance of Electrophoretically Deposited Apatite-Wollastonite/Chitosan Coating on Titanium Implants in Rabbit Tibiae

A bone healing model was used to compare the efficacy of bone healing using uncoated implants and implants coated with a composite coating as described herein. Bone healing of tibial defect in rabbit model was used to evaluate a composite coating of apatite-wollastonite/chitosan on titanium implant. This coating has been developed to overcome the shortcomings, such as implant loosening and lack of adherence, of uncoated titanium implant. An electrophoretic deposition technique was used to coat apatite-wollastonite/chitosan on titanium implant. The present study was designed to evaluate the bone response of coated titanium implants as compared to uncoated titanium implants in an animal model. After implantation period of 14 (Group A), 21 (Group B), 35 (Group C) and 42 days (Group D), bone-implant interface and defect site healing was evaluated using radiography, scintigraphy, histopathology, fluorescence labeling and hematology. Radiography of defect site treated with coated implant suggested expedited healing. Scintigraphy of coated implant site indicated faster bone metabolism than uncoated implant site. Histopathological examination and fluorescence labeling of bone from coated implant site revealed higher osteoblastic activity and faster mineralization respectively. Faster bone healing in the case of coated implant site is attributed to higher cell adhesion on electrostatically charged chitosan surface and apatite-wollastonite assisted mineralization at bone-implant interface. Hematological studies showed no significant difference in hemoglobin, total erythrocyte and leukocyte counts, done using one way-ANOVA, during entire study period. Results presented here show AW/chitosan coated implants impart advantages of faster bone healing, increased mechanical strength and good bone-implant bonding.

Materials and Methods

Preparation of Materials used in Electrophoretic Deposition

Apatite-wollastonite (AW) powder formed by modified sol-gel route (Pattanayak et al., 2006) was used in this study for synthesizing the composite coating. Particle sizing was carried out using a dynamic light scattering (BI-9000 AT Digital Autocorrelator, Brookhaven Instrument, USA) and was found to be 200 nm. Chitosan was obtained from Otto chemicals (98% Deacetylated). Titanium sheet (Manhar metal suppliers, Mumbai, India) of dimension (5 mm×3 mm×0.5 mm) was used as the test substrate. The substrates were etched with 2% hydrofluoric acid (HF) for 1 minute, then rinsed with MilliQ water and air-dried before use.

Deposition Details

Titanium (Ti) test samples were used as both anode and cathode. Distance between the electrodes was maintained at 10 mm. The ceramic particles of apatite-wollastonite were dispersed ultrasonically in ethanol for 30 minutes at 20 Hz (98 kW) in an ultrasonic vibrator. Electrophoretic deposition was performed from suspension of 2 g/L AW particles in ethanol as solvent. The pH of the ceramic suspension was optimized after carrying out repeated experiments and was fixed at pH of 1.6. Suspension of 0.2% of chitosan was prepared in 2% acetic acid solution. Cathodic deposition was performed on titanium (Ti) sheets with a coating area roughly 10 mm×10 mm. Current density was fixed at about 3 mA/cm² to deposit ceramic and about 1 mA/cm² to deposit chitosan. A repeated deposition method was utilized to reduce formation of cracks in the coating. To start with, surface of titanium was coated with thin layer of chitosan followed by three alternate coating cycles of ceramic and chitosan to obtain homogenous composite coating. The last coat of chitosan was repeated two times so as to encapsulate composite coating by polymer thereby preventing the erosion of the final composite coating. The coated and uncoated titanium implants were sterilized with gamma radiation at 20 κGy 30° C. in Gamma Chamber (GC-1200 having ⁶⁰Co as the source, kindly provide by Tata Memorial Hospital, Parel, Mumbai) before implanting. The radiation dose given was according to the standards of the International Atomic Energy Agency (IAEA).

Adhesive Strength of Composite Coatings

For assessing the interfacial adhesive strength of the composite coating on titanium substrate, a standard test method (Tape test—ASTM D 3359-97) was used. This was measured by applying a pressure-sensitive tape (EURO Tape, Century distributors (P) Ltd., India) on the composite coating. Coverage of coated substrate was quantified using Matlab (version 7.1).

Animal Model

The studies presented herein were conducted on 12 healthy mature New Zealand white rabbits of either sex weighing between 1.5-2.5 kg. The experimental protocol was approved by Institutional Animal Ethics Committee as per the guidelines of the Committee for the Purpose of Control and Supervision of Experiments on Animals (CPCSEA), Ministry of Social Justice and Empowerment, Government of India.

Methodology

The rabbits were randomly divided into four groups: Group A (14 days implantation period), Group B (21 days implantation period), Group C (35 days implantation period) and Group D (42 days implantation period), each consisting of three rabbits. Preoperatively each rabbit was kept off feed for a period of 3 hours before induction of anesthesia. Anesthesia was induced by injecting combination of xylazine (7 mg/kg) (intas pharma Ltd, Ahemdabad, Gujarat, India) and ketamine (60 mg/kg) (Themis Medicare Ltd, Vapi, Gujarat, India) intramuscularly. The medial part of both tibiae were shaved and scrubbed. Skin of both legs was scrubbed routinely with savlon (Johnson and Johnson) solution prior to surgery. Every rabbit received two implants, apatite-wollastonite/chitosan coated as test in right tibia and uncoated as control in left tibia.

After the anesthesia, 20 mm longitudinal skin incision was made on medial surface of the tibia on dorsal following proper draping of the site. Subcutaneous tissue and periosteum was separated gently from cortical bone. An appropriate defect size of 5 mm length×1.5 mm width was made using an orthopedic hand drill machine with drill bit size 1.5 mm, under constant irrigation with sterile normal saline to avoid thermal necrosis. The titanium implants used in the study were approximately 1 mm thick. Therefore, it was essential to use a slot which was not much greater than that, hence, the choice of a 1.5 mm drill bit. The periosteum and subcutaneous tissue were sutured with chromic catgut no. 3-0 with simple interrupted sutures. Skin was sutured by nylon using horizontal mattress sutures.

The surgical wound was cleaned with Povidone Iodine (5%) and dressed with Nitrofurazone ointment. Enrofloxacin (5mg/kg body weight intramuscularly) injections were given twice daily for seven days, in order to prevent post operative infection. Meloxicam (0.1-0.2 mg/kg body weight) injections were administered intramuscularly for three days postoperatively as an anti-inflammatory analgesic. Sutures were removed on day 10.

Parameters Studied

Clinical Signs

Rabbits were observed for abnormality in gait. The period taken for normal weight bearing and ambulation were critically observed in all groups of rabbit. The operated on limbs were examined for complications like swelling, sepsis or pain during the postoperative period.

Gross Observations

At the termination of the study, the test bones were removed after euthanizing the rabbit and were observed for soft tissue reaction around the implant, adhesions, changes in the bone at the site of contact with the implant and status of the bone.

Plane Radiography

Lateral and anterior-posterior radiograph of entire length of tibia were taken preoperatively and immediately after the surgery. Subsequently, radiography of each bone was done on day 14, 21, 35 and 42 postoperatively in group A, B, C and D, respectively. The radiographs were observed for size of periosteal callus, bone healing and complications like complete fracture of bones and osteomyelitis if any.

Scintigraphy

Bone Scintigraphy of 4 rabbits one of each group A, B, C and D was performed to evaluate bone metabolism at coated and uncoated titanium implants site. A reliable uptake accessed at titanium implant and positive control was studied to determine the acceptance/rejection on circulation maintained at defect site. ^{99m}Tc-methylene diphosphonate (^{99m}Tc-MDP) was used for in-vivo imaging of the defect and its comparison with contra lateral control. 1 mCi/37 MBq of ^{99m}Tc was administered and accessed for perfusion, tissue uptake immediately after post administration and at 3 hrs post injection (PI). Acquisition of image was done at 140 KeV at 20% window. Dynamic images were acquired in 64×64 matrix for 1 minute. Static images were acquired in 256×256 matrix for 150 Kct. Delayed static images were acquired in 256×256 matrix at 3 hrs post injection. Comparative radiotracer uptake analysis was done by using comparable Rol analysis program on eNTEGRA work station.

Histopathological Studies

The histopathological examination of the bone was done to evaluate the cellular reactions of the host bone to the implant. The bones from the site of fracture were obtained by cutting it in small pieces. The bone pieces were washed thoroughly with normal saline and fixed in 10% Formalin for 7 days. Subsequently bone pieces were decalcified in 5% nitric acid and were checked regularly for status of decalcification. Once the bone pieces become flexible, transparent, and easily penetrable by pins, they were considered as completely decalcified. The tissues were processed in routine procedure and 4 μm thin sections were cut and stained with Haemotoxylin and Eosin.

Fluorescence Labeling

Oxytetracycline dehydrate (50-60 mg/kg body weight) was injected deep intramuscularly on days 7 and 10 postoperatively to each rabbit of group A, days 15 and 18 of group B, days 27 and 30 of group C, and days 35 and 38 of group D for labeling the new bone growth. A thin bone section from the site of bone defect was obtained by grinding thick bone section on coarse grinding paper and was observed under a fluorescence microscope.

Hematological Studies

The following hematological parameters were studied as per the method described by Schalm et al. (1975). Blood samples of 3 ml were collected preoperatively (day 0) and after days 7 and 14 postoperatively from all the animals of group A, days 0, 7, 14 and 21 in group B, on days 0, 7, 14 and 35 in group C and days 0, 7, 14 and day 42 in group D. The following hematological parameters were studied: hemoglobin (Hb), total erythrocyte count (TEC), and total leukocyte count (TLC).

Results

Clinical Signs

Xylazine (10 mg/kg) and ketamine (50 mg/kg) used to induce and maintain the anesthesia for creation of bone defect was found sufficient. None of the animals showed any sign of untoward reaction during the surgical procedure. All the rabbits recovered completely within 30-60 minutes and started feeding on Lucerne grass. None of the rabbits showed any abnormality in their gait or posture. Pain was not evident following surgery in the limbs. There was not any swelling or exudation from the wound, and no other complication of wound healing was recorded in any rabbit in any of the groups. Daily dressing of the wound and antibiotic injection resulted in normal wound healing in all animals. The surgical wound healed completely on day 7 post-operatively, and sutures were removed on day 8 following surgery.

Gross Observations

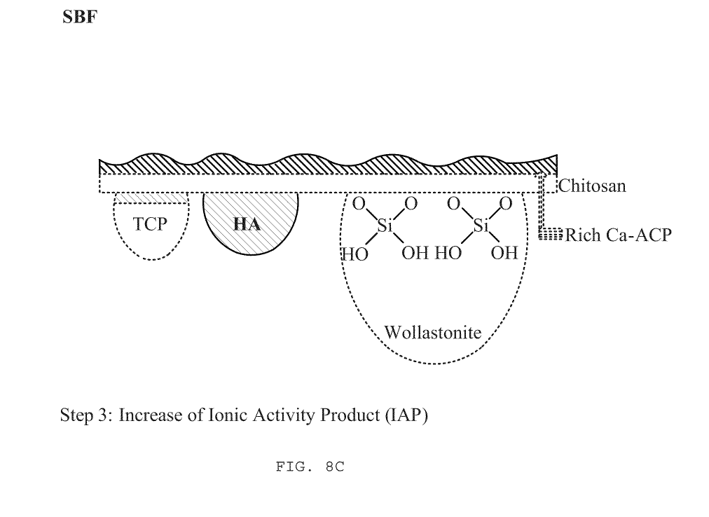

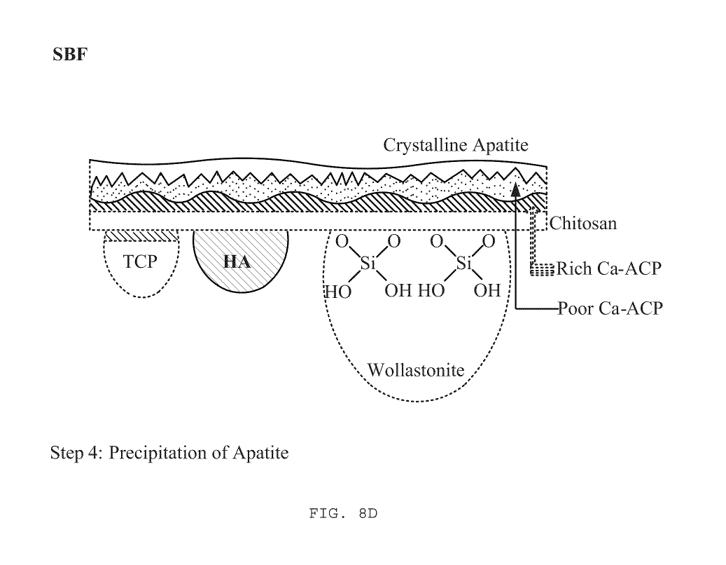

Gross observations performed on all groups at the indicated time intervals showed both implants were well fixed into the host bone. In group A (14 days), soft tissue adhesion was found to be more prominent at the defect site treated with uncoated implant. The border defect site at uncoated implant was clear and defined, whereas defect site was slightly irregular in the case of the coated implant (see FIGS. 10A and 10B). Healing was incomplete at both the defect sites which also was observed in radiographic as well as histopathological findings. In group B (21 days), border of defect site treated with coated implant shows irregular mass of hard bony tissue completely filling the defect. Also, the redness was more prominent near the defect site treated with uncoated titanium implant (see FIGS. 10C and 10D). In group C (35 days), callus formed at defect site of the coated implant seems to be covering uniformly while uncoated implant site shows prominent defect site opening surrounded by reddened patches (see FIGS. 11A and 11B). In group D, red defect site is still visible at uncoated implant site but coated site shows complete healing of defect site resembling host bone (see FIGS. 11C and 11D). So healing rate observed was faster at coated implant defect site as compare to uncoated implant defect site.

Radiography

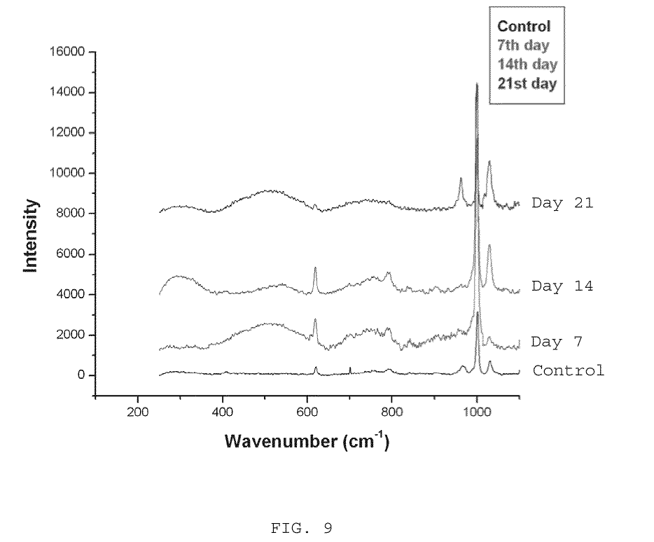

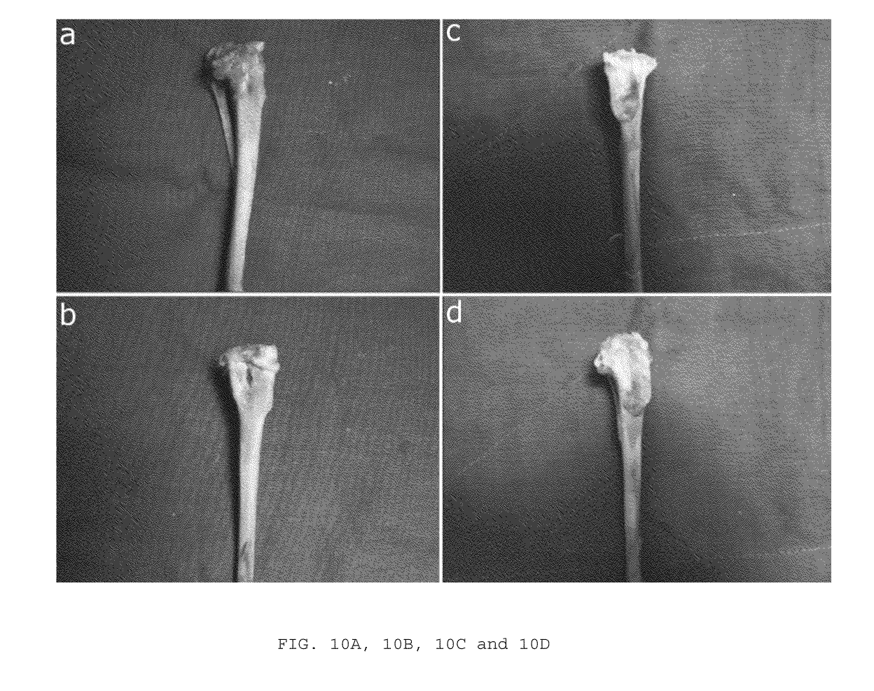

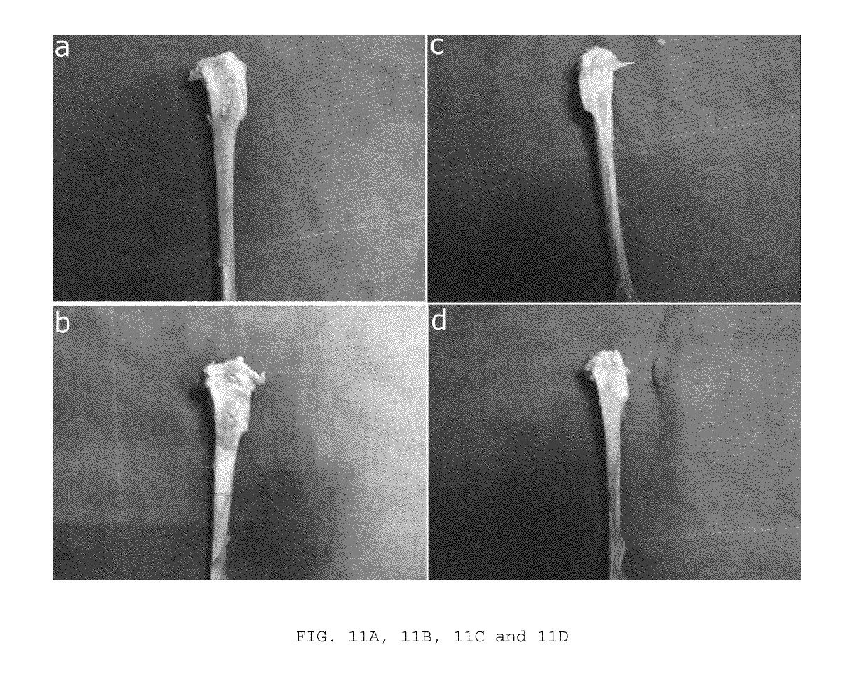

The radiograph taken immediately after creation of bone defect clearly demonstrated radiolucent shadow around the both coated and uncoated titanium implants. Radiograph taken on day 14 (Group A) at both the defect site showed that the implants remained seated at original site with no proximal or distal shift. At 14 days, the defect sites treated with both coated and uncoated titanium implants appeared radiolucent however the area around the defect site implanted with apatite-wollastonite/chitosan coated titanium implant was slightly more radiopaque as compared to that treated with uncoated titanium (see FIGS. 12A-12D).

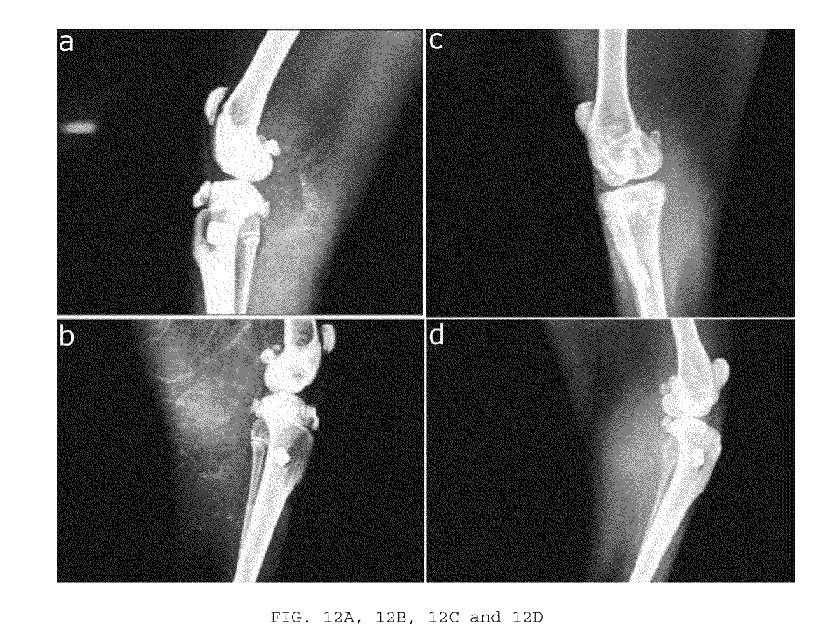

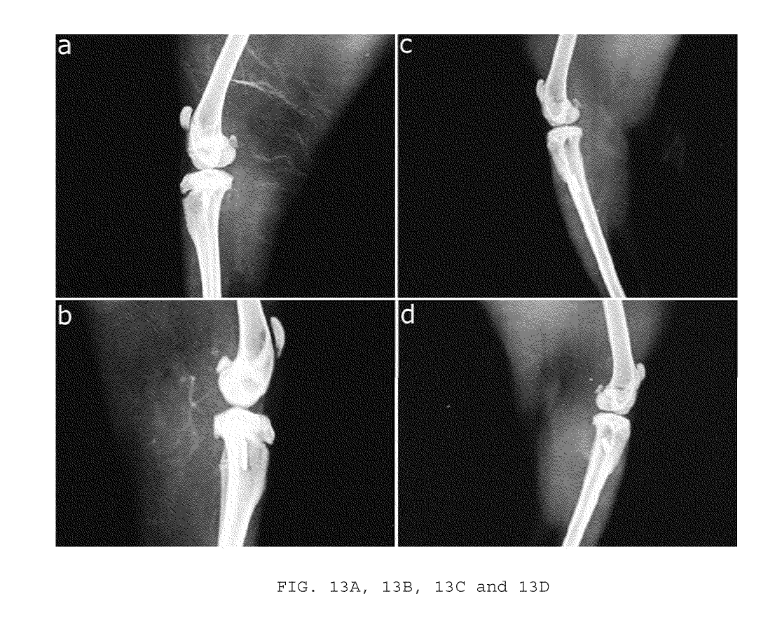

Radiographs taken on day 21 (Group B) confirmed that formation of immature bone was progressive. The defect site treated with coated titanium implant in FIG. 12C showed good and clearly defined radiodense area. This could be due to formation of new bone growth (e.g., trabeculae). The defect site treated with uncoated titanium implant (see FIG. 12D) appeared occupied by the radiopaque callus indicating initiation of osteogenesis at this defect site also. Radiographs taken on day 35 (Group C) showed signs of progressive periosteal healing; however, complete remodeling was not observed. The defect site treated with uncoated titanium implant shows filling of the bone defect with immature woven bone as an radiopaque area at the defect site (see FIG. 13B). A mild spot of radiolucent area was observed at the center of the defect, while the periosteal healing was not clearly visible, demonstrating progressive healing. It was observed that at some places the radiodensity at the defect site treated with coated titanium was nearly comparable to that of the host bone (see FIG. 13A). This indicated faster and progressive bone healing at the defect site treated with coated titanium. Radiographs at day 42 (Group D) showed complete remodeling of the defect site treated with coated titanium implant (see FIG. 4C). The radio-opacity at the defect site treated with coated a coated titanium implant was comparable to that of the host bone.

Scintigraphy

TABLE 1
Counts per pixel in scintigraphy for coated and uncoated titanium implant

| | Right Limb(coated implant) | Left Limb (uncoated implant) |
|---|---|---|
| Group | Counts per pixel | Counts per pixel |
| A | 124.58 | 68.55 |
| B | 475.73 | 168.95 |
| C | 177.93 | 212.82 |
| D | 111.75 | 171.96 |

The counts per pixel of both coated and uncoated titanium are given in the table 1. The counts at defect sites treated with coated titanium were significantly higher initially on days 14 and 21 post-operatively as compared to the defect sites treated with uncoated titanium. The observations were suggestive of initially higher uptake of ^{99m}Tc-MDP at the defect site treated with coated titanium implant, due to faster bone metabolism, than at the defect sites treated with uncoated titanium implant. These count subsequently decreased, suggesting callus organization and reorganization and progressive osteogenesis.

Histopathological Studies

In Group A, moderate infiltration of fibrous connective tissue (FCT) was observed in the case of defect sites treated with uncoated titanium implant (see FIG. 14B). Moderate infiltration of resting cartilage (RC) was observed at uncoated implant sites while diffuse infiltration of RC was seen at coated implant sites, indicating initiation of osteogenesis (see FIG. 14A). Moderate focal foci of hypertrophy of chondrocytes (HC) were observed around uncoated implant sites, whereas multifocal foci of HC at coated implant sites indicate stacking of chondrocytes leading to calcification. Extensive foci of calcification of chondrocytes (CC) are seen at coated implant sites while minimal CC are seen at uncoated implant sites. In Group B (see FIG. 14D), mild infiltration FCT and mild HC were observed at uncoated implant sites. Also, the initiation of CC was followed by mild formation of bone trabeculae (BT). At coated implant sites (see FIG. 14C), extensive calcification and formation of BT indicated cancellous bone ossification.

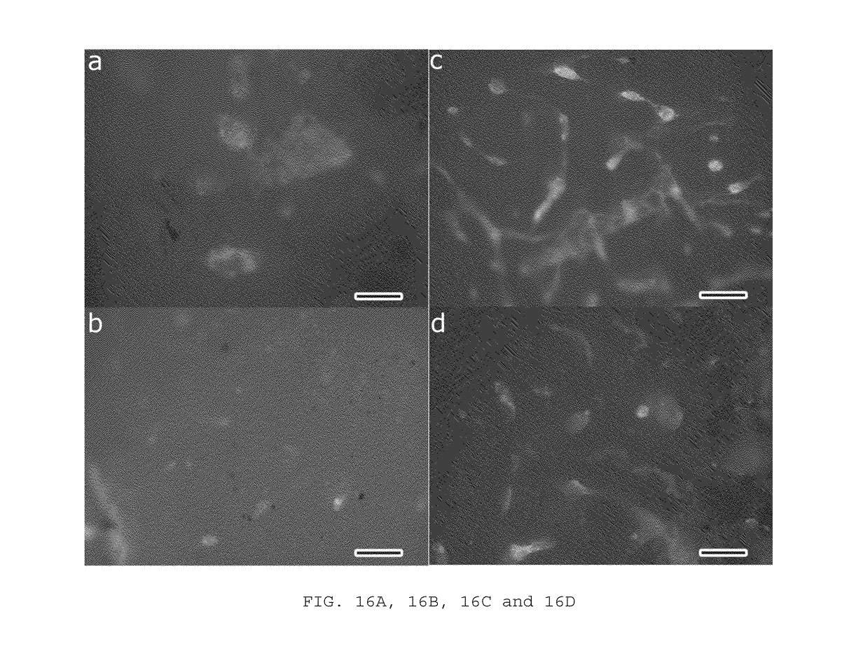

In Group C, mild diffuse formation of BT at coated implant sites signified low osteoblastic activity due to completion of new bone formation and extensive osteoclastic activity as compared to uncoated implant sites. Mild lamellar bone (LB) formation, proliferation of blood vessels and presence of bone marrow is clearly visible at coated implant site (see FIG. 15A). In Group D, extensive formation of lamellar bone with complete bone remodeling can be seen at coated implant sites (see FIG. 15B). Also, better Haversian systems with presence of osteocytes in lacunae were observed. New compact bone is in direct contact with implant having no soft tissues in between, indicates complete and faster healing than at uncoated implant sites.

Fluorescence Labeling Studies

Coated implant sites of Group A showed a few foci of mild diffused green spots indicating the initialization of mineralization, while uncoated implant sites showed green background with no mineralization spots. In Group B (see FIG. 16A), more intense segregated golden spots at the coated implant sites showed extensive mineralization due to the calcification of cartilage. Coated implant sites in Group C showed gold-green fluorescence from the integrated mineralized structure of immature bone, while uncoated implant sites still show segregated diffused gold spots. A well organized pattern of fluorescent labeling at the coated implant sites in Group D (see FIG. 16C) showed extensive formation of lamellar bone, indicating completion of the bone remodeling process, while mineralization was still under progress in the case of uncoated implant sites (see FIG. 16D).

Hematological Studies

In hemoglobin estimation, no significant difference (p>0.05; ANOVA) was found in the total Hb count levels between the groups. There was no loss of blood during either surgery or postoperative care. Further, the animals remained healthy during period of study. No significant differences (p>0.05; ANOVA) in total erythrocyte count and leukocyte count levels were seen between any of the groups until the completion of the experiment.

Adhesive Strength of Composite Coatings

The interfacial adhesive strength of the composite coating on the titanium substrate was measured using a standard test method (tape test—ASTM D 3359-97). This was done by applying a pressure-sensitive tape (EURO Tape) over cuts made on the coating. The coated area removed was found to be 66% in the ceramic coating and 21% in the composite coating. The adhesion test was evaluated using a scale of 0 to 5 with 0 corresponding to very poor and 5 to very good adhesion. Classification of the coating done on the basis of the standard chart showed that with polymer the coating was in the 2B class and without polymer coating it was in the OB class.

Discussion

A systematic study presented herein was done on a rabbit model to evaluate the bone healing performance of a AW/Chitosan coating. A xylazine and ketamine combination for the induction and maintenance of anesthesia in New Zealand rabbits was used successfully herein. None of the animals showed any sign of untoward reactions during the surgical procedure. Pain was not evident following surgery in the limbs. All the rabbits got up following recovery and were comfortable in the cage, with no sign of abnormality in the gait and posture.

Radiography was done to identify the degree of new bone formation around the coated and uncoated implants. Radiographic analysis of metaphysis parts of the tibiae showed that the implants were clearly detectable as radiopaque areas in all specimens of both groups, and the implants remained seated at the original sites with no proximal or distal shift, indicating good interference fit. The presence of radiolucent areas surrounding the implants was observed for all groups. Radiographs of Group A (14 days) showed significant portions of radiolucent area around coated and uncoated implants which was due to the formation of cartilaginous tissue, also seen in histopathological results for group A. A radiograph of the left tibia of a rabbit from Group B (21 days) showed radiolucent patches around the uncoated implant site, while the right tibia showed a moderate radiopaque region around the coated implant. This can be explained by the formation of trabecular bone and extensive calcification leading to the radiopaque region at the coated implant site. Group C (35 days) and Group D (42 days) showed a progressive increase in the radiodense area and eventual disappearance of radiolucent line between the coated implant and host bone, indicating good bone-implant integration. Also, bone bonding with the implants was better in the case of coated implants, due to the formation of an apatite layer in between the new bone and the implant surface. However, group D (42 days) uncoated implant sites still showed the presence of a radiolucent shadow, suggesting incomplete healing.

Bone Scintigraphy, a bone metabolism imaging technique, measures the distribution of a radiolabelled phosphorous compound (^{99m}Tc-MDP) around the defect site, which is dependent on bone metabolism rate and blood flow. Table 1 shows Group A (14 days) had nearly double the counts per pixel at coated implant sites than at uncoated implant sites. The higher concentration of radionuclide in Group A at coated sites is due to the initiation of calcification and partly due to new apatite layer formed. Group B (21 days) shoed the highest count per pixel for coated implant sites, which is indicative of extensive osteoblastic activity and higher metabolism, but uncoated implant sites showed the highest metabolic activity in Group C (35 days), which represented comparatively delayed metabolism. The progressive decrease in radionuclide concentration for coated and uncoated implants was due to a decrease in osteoblastic activity, increase in osteoclastic activity and gradual remodeling.

Histopathology of the bone section taken from the defect site was used to study the bone regeneration and interaction of AW/Chitosan coated implants and titanium implants with surrounding tissues. In group A (14 days), coated implant sites shoed the presence of multifocal foci of HC with CC, while uncoated implant sites showed mild foci of HC with the presence of FCT. Chitosan may improve initial attachment of cells due to electrostatic interactions, which can be supported by an increase in adhesion of chondrocytes at coated implant sites leading to their hypertrophy and calcification. In group B (21 days), formation of BT and extensive mineralization can be seen at coated implant sites, while uncoated implant sites showed traces of mineralization and CC. The extensive mineralization rate at coated implant sites, which was aided by the bioactive property of AW to form apatite layer, was also supported by intense fluorescence in group B (see FIG. 16B). Groups C (35 days) and Group D (42 days) showed the usual transition of immature to mature bone formation.

No significant difference was found in levels of hemoglobin, erythrocyte and leukocyte counts until the completion of the study. The rabbits in this study were given an anti-inflammatory analgesic following surgery, so the leukocyte count would have been remained unaltered during the postoperative care. Further, no complication of wound healing was seen in any of the groups

Conclusions

The study presented herein suggests that AW/Chitosan coated titanium implants help in faster bone healing than uncoated titanium implants. Incorporation of chitosan fibers proved to increase interfacial adhesive strength and osteoconductive properties of the composite coating. Within the limitations of the study presented herein, AW/Chitosan seems to be a useful material for coating prosthetic devices to be inserted in bone.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

The present disclosure is not to be limited in terms of particular embodiments described in this disclosure, which are illustrations of various aspects. Many modifications and variations can be made without departing from the spirit and scope of the disclosure, as will be apparent to those skilled in the art. Functionally equivalent methods and apparatuses within the scope of the disclosure, in addition to those enumerated herein, will be apparent to those skilled in the art from the foregoing descriptions. Such modifications and variations fall within the scope of the appended claims. The present disclosure is to be limited only by the terms of claims (e.g., the claims appended hereto) along with the full scope of equivalents to which such claims are entitled. It is to be understood that this disclosure is not limited to particular methods, reagents, compounds compositions or biological systems, which can, of course, vary. It is also to be understood that terminology used herein is for the purpose of describing particular embodiments only, and is not necessarily limiting.

With respect to the use of substantially any plural and/or singular terms herein, those having skill in the art can translate from the plural to the singular and/or from the singular to the plural as is appropriate to the context and/or application. Various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.).

The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Thus, it should be understood that although the present technology has been specifically disclosed by representative embodiments and optional features, modification and variation of the concepts herein disclosed may be resorted to by those skilled in the art, and such modifications and variations are considered within the scope of this technology. As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not limiting, with the true scope and spirit of certain embodiments indicated by the following claims.

What is claimed is:

1. A method, comprising:
contacting a support material with a bioactive polymer under a first set of electrophoresis conditions that include applying an electric current of less than 5 mA/cm2, thereby depositing the bioactive polymer onto the support material;
contacting the support material with bioceramic particles under a second set of electrophoresis conditions, thereby depositing the bioceramic particles onto the support material; and
alternating the depositing of the bioactive polymer and the bioceramic particles onto the support material in a predetermined number of cycles, thereby preparing a coated support material.

2. The method of claim 1, wherein the bioactive polymer and bioceramic particles are deposited in layers.

3. The method of claim 2, wherein the layers have a thickness of about 0.1 micrometers to about 100 micrometers.

4. The method of claim 1, wherein the thickness of the coating is about 5 micrometers to about 500 micrometers.

5. The method of claim 1, wherein the metal comprises titanium.

6. The method of claim 1, wherein the support material is etched.

7. The method of claim 1, wherein the support material is a medical implant.

8. The method of claim 1, wherein the bioactive polymer comprises a polysaccharide.

9. The method of claim 8, wherein the polysaccharide comprises glucose.

10. The method of claim 9, wherein the polysaccharide includes a 2-amino-2-D-glucose polymer.

11. The method of claim 10, wherein the polysaccharide includes chitosan.

12. The method of claim 1, wherein the bioceramic particles comprise apatite and wollastonite.

13. The method of claim 12, wherein the particles are about 200 nanometers in diameter.

14. The method of claim 1, wherein electrophoresis, under the first electrophoresis conditions, is carried out for less than about 5 minutes.

15. The method of claim 1, wherein electrophoresis, under the first electrophoresis conditions, is performed at a constant current.

16. The method of claim 1, wherein electrophoresis, under the second electrophoresis conditions, is performed at a constant current.

17. The method of claim 1, wherein each cycle is repeated between about 2 times and about 100 times.

18. A coated solid material, comprising a support material that includes an exterior surface and a coating adhered to the exterior surface that includes a bioactive polymer and a bioceramic particles, wherein the coated solid material is prepared by a method comprising:
contacting the support material with the bioactive polymer under a first set of electrophoresis conditions that include applying an electric current of less than 5 mA/cm2, thereby depositing the bioactive polymer onto the support material;
contacting the support material with the glass-ceramic particles under a second set of electrophoresis conditions, thereby depositing the bioceramic particles onto the support material; and
alternating the depositing of the bioactive polymer and the bioceramic particles onto the support material in a predetermined number of cycles, thereby preparing a coated support material.

19. A coated material, comprising:
a support material having an exterior surface, a coating adhered to the exterior surface, and cells in association with the coated material, wherein:
the coating comprises a polysaccharide that includes glucose, and the coating comprises bioceramic particles that include apatite and wollastonite.

20. The coated material of claim 19, wherein the coated material is a medical implant.
